(19) United States
(12) Patent Application Publication
(10) Pub. No.: US 2011/0098300 A1
(43) Pub. Date: Apr. 28, 2011

(54) Compounds Comprising A Cyclobutoxy Group

(75) Inventors: Sylvain Celanire, Reignier (FR); Laurent Provins, Brussells (BE); Frederic Denonne, Brussells (BE); Anne Valade, Brussells (BE)

(73) Assignee: UCB PHARMA, S.A., Brussells (BE)

(21) Appl. No.: 12/995,084

(22) PCT Filed: Jun. 2, 2009
(86) PCT No.: PCT/EP09/56758

§ 371 Date: Jan. 5, 2011

(30) Foreign Application Priority Data
Jun. 6, 2008 (EP) 08104281.4

(51) Int. Cl. A61K 31/5377; C07D 211/60; C07D 413/12; A61K 31/4453; A61P 9/10; A61P 25/00; A61P 25/18; A61P 25/28

(52) U.S. Cl. 514/235.5; 546/189; 544/141; 544/130; 514/316

(57) ABSTRACT

The present invention relates to compounds of formula (I) comprising a cyclobutoxy group, processes for preparing them, pharmaceutical compositions comprising said compounds and their use as pharmaceuticals, useful for the treatment and prevention of diseases or pathological conditions of the central nervous system including mild-cognitive impairments, Alzheimer's disease, learning and; memory disorders, cognitive disorders, attention deficit disorder, attention-deficit hyperactivity disorder, Parkinson's disease, schizophrenia, dementia, depression, epilepsy, seizures, convulsions, sleep/wake and arousal/vigilance disorders such as hypersomnia and narcolepsy, pain and/or obesity.

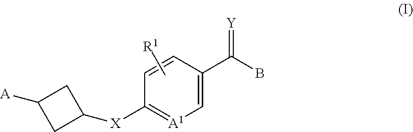

The present invention relates to compounds comprising a cyclobutoxy group, processes for preparing them, pharmaceutical compositions comprising said compounds and their use as pharmaceuticals.

The histamine H₃ receptor has been known for several years and identified pharmacologically in 1983 by Arrang, J. M. et al. (Nature 1983, 302, 832-837). Since the cloning of the human histamine H₃ receptor in 1999, histamine H₃ receptors have been successively cloned by sequence homology from a variety of species, including rat, guinea pig, mouse and monkey.

Histamine H₃-receptor agonists, antagonists and inverse agonists have shown potential therapeutic applications as described in the literature, for example by Stark, H. in Exp. Opin. Ther. Patents 2003, 13, 851-865, and by Leurs R. et al. in Nature Review Drug Discovery 2005, 4, 107-120.

The histamine H₃ receptor is predominantly expressed in the mammalian central nervous system but can also be found in the autonomic nervous system. Evidence has been shown that the histamine H₃ receptor displays high constitutive activity, which activity occurs in the absence of endogenous histamine or of a H₃-receptor agonist. Thus, a histamine H₃-receptor antagonist and/or inverse agonist could inhibit this activity.

The general pharmacology of histamine H₃ receptor, including H₃-receptor subtypes, has been reviewed by Hancock, A. A in Life Sci. 2003, 73, 3043-3072. The histamine H₃ receptor is not only considered as a presynaptic autoreceptor on histaminergic neurons, but also as a heteroreceptor on non-histaminergic neurons (Barnes, W. et al., Eur. J. Pharmacol. 2001, 431, 215-221). Indeed, the histamine H₃ receptor has been shown to regulate the release of histamine but also of other important neurotransmitters, including acetylcholine, dopamine, serotonin, norepinephrin and γ-aminobutyric acid (GABA).

Thus, the histamine H₃ receptor is of current interest for the development of new therapeutics and the literature suggests that novel histamine H₃-receptor antagonists or inverse agonists may be useful for the treatment and prevention of diseases or pathological conditions of the central nervous system including Mild Cognitive Impairment (MCI), Alzheimer's disease, learning and memory disorders, cognitive disorders, attention deficit disorder (ADD), attention-deficit hyperactivity disorder (ADHD), Parkinson's disease, schizophrenia, dementia, depression, epilepsy, seizures or convulsions, sleep/wake disorders, narcolepsy, pain and/or obesity.

H₃-receptor ligands alone or in combination with an acetylcholinesterase inhibitor may also be useful in the treatment of cholinergic-deficit disorders, Mild Cognitive Impairment and Alzheimer's disease as reported by Morisset, S. et al. in Eur. J. Pharmacol. 1996, 315, R1-R2.

H₃-receptor ligands, alone or in combination with a histamine H₁-receptor antagonist may be useful for the treatment of upper airway allergic disorders, as reported by McLeod, R. et al. in J. Pharmacol. Exp. Ther. 2003, 305, 1037-1044.

H₃-receptor ligands, alone or in combination with a serotonine reuptake inhibitor may be useful for the treatment of depression, as reported by Keith, J. M. et al in Bioorg. Med. Chem. Lett. 2007, 17, 702-706.

As described in international patent application WO 02/072093, H₃-receptor ligands alone or in combination with a muscarinic receptor ligand and particularly with a muscarinic M₂-receptor antagonist, may be useful for the treatment of cognitive disorders, Alzheimer's disease, attention-deficit hyperactivity disorder.

H₃-receptor ligands may also be useful in the treatment of sleep/wake and arousal/vigilance disorders such as hypersomnia, and narcolepsy according to Passani, M. B. et al. in Trends Pharmacol. Sci. 2004, 25(12), 618-625.

In general, H₃-receptor ligands, and particularly H₃-receptor antagonists or inverse agonists may be useful in the treatment of all types of cognitive-related disorders as reviewed by Hancock, A. A and Fox, G. B. in Expert Opin. Invest. Drugs 2004, 13, 1237-1248.

In particular, histamine H₃-receptor antagonists or inverse agonists may be useful in the treatment of cognitive dysfunctions in diseases such as Mild Cognitive Impairment, dementia, Alzheimer's disease, Parkinson's disease, Down's syndrome as well as in the treatment of attention-deficit hyperactivity disorder (ADHD) as non-psychostimulant agents (see for example Witkin, J. M. et al., Pharmacol. Ther. 2004, 103(1), 1-20).

H₃-receptor antagonists or inverse agonists may also be useful in the treatment of psychotic disorders such as schizophrenia, migraine, eating disorders such as obesity, inflammation, pain, anxiety, stress, depression and cardiovascular disorders, in particular acute myocardial infarction.

There is therefore a need to manufacture new compounds which can potentially act as H₃-receptor ligands.

Early literature reports (e.g. Ali, S. M. et al., J. Med. Chem. 1999, 42, 903-909 and Stark, H. et al., Drugs Fut. 1996, 21, 507-520) describe that an imidazole function is essential for high affinity histamine H₃-receptor ligands; this is confirmed, for example, by U.S. Pat. Nos. 6,506,756B2, 6,518,287B2, 6,528,522B2 and 6,762,186B2 which relate to substituted imidazole compounds that have H₃-receptor antagonist or dual histamine H₁-receptor and H₃-receptor antagonist activity.

International patent application WO 02/12214 relates to non-imidazole aryloxyalkylamines for the treatment of disorders and conditions mediated by the histamine receptor.

International patent application WO 02/076925 relates to non-imidazole aryl alkylamines compounds as histamine H3 receptor antagonists.

International patent application WO 2004/037800 describes a class of arylalkoxy amine derivatives as H3 ligands.

International patent application WO 2006/136924 describes a class of phenoxycyclobutyl derivatives as H₃-receptor antagonists.

US patent application US 2005/171181 discloses cyclobutyl-arylamines as H₃-receptor modulators.

International patent applications WO 2006/132914 and WO 2007/038074 describe cyclobutyl amine derivatives as H₃-receptor modulators.

International patent application WO 2008/128919 discloses compounds comprising a cyclobutoxy group.

European patent application n° 08001308.9 discloses 4-[(trans-3-piperidin-1-ylcyclobutyl)sulfanyl]benzamide, 4-[(trans-3-piperidin-1-ylcyclobutyl)sulfanyl]benzenecarbothioamide, N-(4-chloropyridin-3-yl)-4-[(trans-3-piperidin-1-ylcyclobutyl)oxy]benzamide and related cyclobutyloxybenzamide as synthetic intermediates involved in the preparation of H₃-receptor ligands.

International patent application n° PCT/EP2009/050719 discloses N-(2-oxoazepan-3-yl)-4-{[trans-3-(piperidin-1-yl)cyclobutyl]oxy}benzamide and N-(3-aminopyridin-4-yl)-4-{[trans-3-(piperidin-1-yl)cyclobutyl]oxy}benzamide and related cyclobutyloxybenzamide as synthetic intermediates involved in the preparation of H₃-receptor ligands.

It has now surprisingly been found that compounds of formula (I) may act as H₃-receptor ligands and therefore may demonstrate therapeutic properties for one or more pathologies mentioned below.

The present invention relates to compounds of formula (I), geometrical isomers, enantiomers, diastereoisomers, pharmaceutically acceptable salts and all possible mixtures thereof,

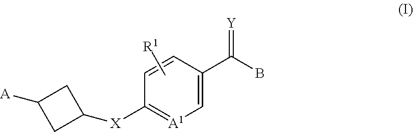

wherein

A is a substituted or unsubstituted amino group which is linked to the cyclobutyl group via an amino nitrogen;

A¹ is CH, C-halogen, C-alkoxy or N;

Y is O or S;

B is a substituted or unsubstituted amino group which is linked to the carbonyl or thiocarbonyl group via an amino nitrogen;

X is O or S; and

R¹ is hydrogen or C₁₋₆ alkyl or halogen or C₁₋₆ alkoxy.

Particularly, the present invention relates to compounds of formula (I) different from N-(2-oxoazepan-3-yl)-4-{[trans-3-(piperidin-1-yl)cyclobutyl]oxy}benzamide.

More particularly, the present invention relates to compounds of formula (I) wherein when X is O and Y is O,

B is different from a group of formula (IX),

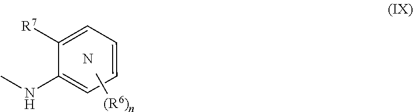

wherein R⁶ is selected from the group comprising or consisting of sulfonyl, amino, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted C₃₋₈ cycloalkyl, substituted or unsubstituted 3-8-membered heterocycloalkyl, acyl, substituted or unsubstituted C₁₋₆-alkyl aryl, substituted or unsubstituted C₁₋₆-alkyl heteroaryl, substituted or unsubstituted C₂₋₆-alkenyl aryl, substituted or unsubstituted C₂₋₆-alkenyl heteroaryl, substituted or unsubstituted C₂₋₆-alkynyl aryl, substituted or unsubstituted C₂₋₆-alkynyl heteroaryl, substituted or unsubstituted C₁₋₆-alkyl cycloalkyl, substituted or unsubstituted C₁₋₆-alkyl heterocycloalkyl, substituted or unsubstituted C₂₋₆-alkenyl cycloalkyl, substituted or unsubstituted C₂₋₆-alkenyl heterocycloalkyl, substituted or unsubstituted C₂₋₆-alkynyl cycloalkyl, substituted or unsubstituted C₂₋₆-alkynyl heterocycloalkyl, alkoxycarbonyl, aminocarbonyl, substituted or unsubstituted C₁₋₆-alkyl carboxy, substituted or unsubstituted C₁₋₆-alkyl acyl, substituted or unsubstituted aryl acyl, substituted or unsubstituted heteroaryl acyl, substituted or unsubstituted C₃₋₈-(hetero)cycloalkyl acyl, substituted or unsubstituted C₁₋₆-alkyl acyloxy, substituted or unsubstituted C₁₋₆-alkyl alkoxy, substituted or unsubstituted C₁₋₆-alkyl alkoxycarbonyl, substituted or unsubstituted C₁₋₆-alkyl aminocarbonyl, substituted or unsubstituted C₁₋₆-alkyl acylamino, acylamino, acylaminocarbonyl, ureido, substituted or unsubstituted C₁₋₆-alkyl ureido, substituted or unsubstituted C₁₋₆-alkyl carbamate, substituted or unsubstituted C₁₋₆-alkyl amino, substituted or unsubstituted C₁₋₆-alkyl sulfonyloxy, substituted or unsubstituted C₁₋₆-alkyl sulfonyl, substituted or unsubstituted C₁₋₆-alkyl sulfinyl, substituted or unsubstituted C₁₋₆-alkyl sulfanyl, substituted or unsubstituted C₁₋₆-alkyl sulfonylamino, aminosulfonyl, substituted or unsubstituted C₁₋₆-alkyl aminosulfonyl, hydroxy, substituted or unsubstituted C₁₋₆-alkyl hydroxy, phosphonate, substituted or unsubstituted C₁₋₆-alkyl phosphonate, halogen, cyano, carboxy, oxo and thioxo;

R⁷ is Cl or NH₂; and

n is equal to 0, 1, 2 or 3.

Even more particularly, the present invention relates to compounds of formula (I) wherein B is an amino group different from —NH₂.

In a particular embodiment, the present invention relates to compounds of formula (I), geometrical isomers, enantiomers, diastereoisomers, pharmaceutically acceptable salts and all possible mixtures thereof,

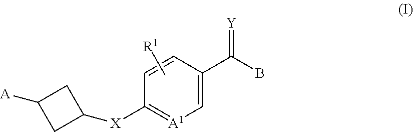

wherein

A is a substituted or unsubstituted amino group which is linked to the cyclobutyl group via an amino nitrogen;

A¹ is CH, C-halogen, C-alkoxy or N;

Y is O or S;

B is a substituted or unsubstituted cyclic amino group which is linked to the carbonyl or thiocarbonyl group via an amino nitrogen;

X is O or S; and

R¹ is hydrogen or C₁₋₆ alkyl or halogen or C₁₋₆ alkoxy.

The term “alkyl”, as used herein, is a group which represents saturated, monovalent hydrocarbon radicals having straight (unbranched) or branched moieties, or combinations thereof, and containing 1-8 carbon atoms, preferably 1-6 carbon atoms; more preferably alkyl groups have 1-4 carbon atoms.

“Alkyl” groups according to the present invention may be unsubstituted or substituted. Examples of alkyl groups according to the present invention are methyl, ethyl, n-propyl and isopropyl. Preferred alkyl group is isopropyl.

“Alkyl” groups may be substituted by one or more substituents including halogen.

The term “halogen”, as used herein, represents a fluorine, chlorine, bromine, or iodine atom. Preferred halogen according to the present invention is fluorine.

The term “hydroxy”, as used herein, represents a group of formula —OH.

The term “hydrogen”, as used herein encompasses all isotopic forms of hydrogen atom

The term “C₁₋₆-alkyl hydroxy”, as used herein, refers to an alkyl as defined above substituted by one or more “hydroxy”.

The term “C₃₋₈ cycloalkyl”, as used herein, represents a monovalent group of 3 to 8 carbon atoms derived from a saturated cyclic hydrocarbon. Examples of C₃₋₈ cycloalkyl groups according to the present invention are cyclopropyl, cyclobutyl, cyclopentyl, cyclohexyl and cycloheptyl. Preferred C₃₋₈ cycloalkyl is cyclobutyl.

The term “C₃₋₈ cycloalkenyl”, as used herein, represents a monovalent group of 3 to 8 carbon atoms derived from a partially unsaturated cyclic hydrocarbon.

The term “C₁₋₆-alkyl cycloalkyl”, as used herein, refers to a C₁₋₆ alkyl having a cycloalkyl substitutent as defined here above.

The term “alkylene”, as used herein, represents a group of formula —(CH₂)ₓ— in which x is comprised between 2 and 6, preferably comprised between 3 and 6.

The term “methylene” as used herein represents a group of formula —CH₂—.

The term “C₂₋₆ alkenyl” refers to alkenyl groups preferably having from 2 to 6 carbon atoms and having at least 1 or 2 sites of alkenyl unsaturation.

The term “C₂₋₆ alkynyl” refers to alkynyl groups preferably having from 2 to 6 carbon atoms and having at least 1 to 2 sites of alkynyl unsaturation.

The term “aryl” as used herein, refers to an unsaturated aromatic carbocyclic group of from 6 to 14 carbon atoms having a single ring (e.g. phenyl) or multiple condensed rings (e.g. naphthyl). The “aryl” groups may be unsubstituted or substituted by 1 to 4 substituents independently selected from halogen, C₁₋₄ alkyl or C₁₋₄ alkoxy as defined herein.

The term “C₁₋₆-alkyl aryl”, as used herein, refers to a C₁₋₆ alkyl having an aryl substituent as defined hereabove.

The term “heteroaryl” as used herein represents an aryl group as defined here above wherein one or more of the carbon atoms have been replaced by one or more heteroatoms selected from O, S or N.

The term “C₁₋₆-alkyl heteroaryl” refers to a C₁₋₆ alkyl having a heteroaryl substituent as defined here above.

The term “C₂₋₆-alkenyl aryl”, as used herein, refers to a C₂₋₆ alkenyl substituted by an aryl as defined here above.

The term “C₂₋₆-alkenyl heteroaryl”, as used herein, refers to a C₂₋₆ alkenyl substituted by a heteroaryl as defined here above.

The term “C₂₋₆-alkynyl aryl”, as used herein, refers to a C₂₋₆ alkynyl substituted by an aryl as defined here above.

The term “C₂₋₆-alkynyl heteroaryl”, as used herein, refers to a C₂₋₆ alkynyl substituted by a heteroaryl as defined here above.

The term “alkoxy”, as used herein, represents a group of formula —OR^{a} wherein R^{a} is an alkyl or an aryl group, as defined above.

The term “C₁₋₆-alkyl alkoxy”, as used herein, refers to a C₁₋₆ alkyl group having an alkoxy substituent as defined hereabove.

The term “carbonyl”, as used herein represents a group of formula —C(═O)—.

The term “thiocarbonyl”, as used herein represents a group of formula —C(═S)—.

The term “acyl”, as used herein, represents a group of formula —C(═O)R^{b} wherein R^{b} is C₁₋₆ alkyl, C₁₋₆-alkyl hydroxy, C₁₋₆-alkyl amino or C₁₋₆-alkyl aminocarbonyl, as defined herein.

The term “C₁₋₆-alkyl acyl” as used herein refers to a C₁₋₆ alkyl having an acyl substituent as defined here above.

The term “3-8-membered heterocycloalkyl” as used herein represents a C₃₋₈ cycloalkyl as defined here above wherein one, two or three carbon atoms are replaced by one, two or three atoms selected from O, S or N. The heterocycloalkyl may be unsubstituted or substituted by any suitable group including, but not limited to, one or more, typically one, two or three, moieties selected from alkyl, halogen, hydroxy, carbonyl, amino, C₃₋₈ cycloalkyl and C₁₋₆-alkyl hydroxy as defined herein.

Examples of 3-8-membered heterocycloalkyl according to the present invention are morpholinyl, 4,4-difluoropiperidinyl, piperidinyl, 4-isopropylpiperazinyl, 4-hydroxypiperidinyl, thiomorpholinyl, 1,1-dioxidothiomorpholinyl, pyrrolidinyl, 3,3-difluoropyrrolidinyl, 4-acetylpiperazinyl, 2-methylpyrrolidinyl, (2S)-2-methylpyrrolidinyl, (2R)-2-methylpyrrolidinyl, 3-hydroxyazetidinyl, 4-oxoimidazolidinyl, 2-(methoxymethyl)pyrrolidinyl, 4-hydroxyisoxazolidinyl, 1,3-thiazolidinyl, 3-oxopyrazolidinyl, 1,4-oxazepanyl, 4-carbamoylpiperidinyl and 4-oxopiperidinyl.

The term “C₃₋₈-(hetero)cycloalkyl acyl” as used herein refers to a 3-8-membered heterocycloalkyl group having an acyl substituent as defined here above. An example of a “C₃₋₈-(hetero)cycloalkyl acyl” is 4-acetylpiperazinyl.

The term “C₁₋₆-alkyl heterocycloalkyl”, as used herein, refers to a C₁₋₆ alkyl substituted by a heterocycloalkyl as defined here above.

The term “C₂₋₆-alkenyl heterocycloalkyl”, as used herein, refers to a C₂₋₆-alkenyl substituted by a heterocycloalkyl as defined here above.

The term “C₂₋₆-alkynyl cycloalkyl”, as used herein, refers to a C₂₋₆ alkynyl substituted by a cycloalkyl as defined here above.

The term “C₂₋₆-alkynyl heterocycloalkyl”, as used herein, refers to a C₂₋₆-alkynyl substituted by a heterocycloalkyl as defined here above.

The term “aryl acyl” as used herein refers to an aryl group having an acyl substituent as defined here above.

The term “heteroaryl acyl” as used herein refers to an heteroaryl group having an acyl substituent as defined here above.

The term “amino group”, as used herein, represents a group of formula —NR^{c}R^{d} wherein R^{c} and R^{d} are independently hydrogen, “C₁₋₆ alkyl”, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl cycloalkyl” or “C₁₋₆-alkyl heterocycloalkyl” groups; or a cyclic group of formula —NR^{c}R^{d} wherein R^{c} and R^{d} are linked together with N, preferably to form a 3 to 8 membered, more preferably a 5 to 7 membered heterocycloalkyl, as defined herein.

Examples of “amino group” according to the present invention are morpholin-4-yl, 4,4-difluoropiperidin-1-yl, piperidin-1-yl, 4-isopropylpiperazin-1-yl, 4-hydroxypiperazin-1-yl, thiomorpholin-4-yl, pyrrolidin-1-yl, 1,1-dioxidothiomorpholin-4-yl, 3,3-difluoropyrrolidin-1-yl, 3-hydroxyazetidin-1-yl, 4-acetylpiperazin-1-yl, (3-chloropyridin-4-yl)amino, (2,2,2-trifluoroethyl)amino, 2-methylpyrrolidin-1-yl, (2S)-2-methylpyrrolidin-1-yl, (2R)-2-methylpyrrolidin-1-yl, 4-oxoimidazolidin-1-yl, 2-(methoxymethyl)pyrrolidin-1-yl, 4-hydroxyisoxazolidin-2-yl, 1,3-thiazolidin-3-yl, 3-oxopyrazolidin-1-yl, 1,4-oxazepan-4-yl, 4-carbamoylpiperidin-1-yl and 4-oxopiperidin-1-yl.

The term “C₁₋₆-alkyl amino”, as used herein, represents a C₁₋₆ alkyl group substituted by an amino group as defined above.

The term “carbamoyl” as used herein refers to a group of formula —C(O)NH₂.

The term “aminocarbonyl” as used herein refers to a group of formula —C(O)NR^{c}R^{d} wherein R^{c} and R^{d} are as defined here above for the amino group.

The term “C₁₋₆-alkyl aminocarbonyl” as used herein, refers to a C₁₋₆ alkyl substituted by an aminocarbonyl as defined hereabove.

The term “C₃₋₈-cycloalkyl amino”, as used herein, represents a C₃₋₈ cycloalkyl group substituted by an amino group as defined above.

The term “acylamino”, as used herein refers to a group of formula —NR^{c}C(O)R^{d} wherein R^{c} and R^{d} are as defined hereabove for the amino group.

The term “C₁₋₆-alkyl acylamino”, as used herein refers to a C₁₋₆ alkyl substituted by an acylamino as defined hereabove.

The term “carboxy”, as used herein represents a group of formula —COOH.

The term “C₁₋₆-alkyl carboxy”, as used herein refers to a C₁₋₆ alkyl substituted by a carboxy group.

The term “cyano”, as used herein represents a group of formula —CN.

The term “alkoxycarbonyl” refers to the group —C(O)OR^{g} wherein R^{g} includes “C₁₋₆ alkyl”, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl” or “C₁₋₆-alkyl heteroaryl”, “C₂₋₆-alkyl cycloalkyl”, “C₁₋₆-alkyl heterocycloalkyl”. Examples of alkoxycarbonyl according to the present invention are tert-butoxycarbonyl and methoxycarbonyl.

The term “C₁₋₆-alkyl alkoxycarbonyl” refers to a C₁₋₆ alkyl having an alkoxycarbonyl as defined here above as substituent.

The term “acyloxy” as used herein refers to a group of formula —OC(═O)R^{b} wherein R^{b} is as defined here above for acyl group.

The term “C₁₋₆-alkyl acyloxy” as used herein refers to a C₁₋₆ alkyl substituted by an acyloxy as defined here above.

The term “acylaminocarbonyl” refers to the group —C(O)NR^{h}C(O)Rⁱ wherein R^{h} and Rⁱ represent independently hydrogen, “C₁₋₆ alkyl”, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl” or “C₁₋₆-alkyl heteroaryl”, “C₂₋₆-alkyl cycloalkyl”, “C₁₋₆-alkyl heterocycloalkyl”.

The term “ureido” as used herein refers to a group of formula —NRⁱC(O)NR^{c}R^{d} wherein Rⁱ is as defined here above for R^{c} or R^{d}, and R^{c} and R^{d} are as defined here above for the amino group. Rⁱ is typically hydrogen or C₁₋₄ alkyl.

The term “C₁₋₆-alkyl ureido” as used herein refers to a C₁₋₆ alkyl substituted by an ureido as defined here above.

The term “carbamate”, as used herein, refers to a group of formula —NR^{c}C(O)OR^{d} wherein R^{c} and R^{d} are as defined here above for the amino group.

The term “C₁₋₆-alkyl carbamate” as used herein refers to a C₁₋₆ alkyl substituted by a carbamate as defined here above.

The term “oxo” as used herein refers to ═O.

The term “thioxo” as used herein refers to ═S.

The term “sulfonyl” as used herein refers to a group of formula “—SO₂—R^{k}” wherein R^{k} is selected from H, “aryl”, “heteroaryl”, “C₁₋₆ alkyl”, “C₁₋₆ alkyl” substituted with halogens, e.g., an —SO₂—CF₃ group, “C₂₋₆ alkenyl”, “C₂₋₆ alkynyl”, “C₃₋₈ cycloalkyl”, “heterocycloalkyl”, “aryl”, “heteroaryl”, “C₁₋₆-alkyl aryl” or “C₁₋₆-alkyl heteroaryl”, “C₂₋₆-alkenyl aryl”, “C₂₋₆-alkenyl heteroaryl”, “C₂₋₆-alkynyl aryl”, “C₂₋₆-alkynyl heteroaryl”, “C₁₋₆-alkyl cycloalkyl” or “C₁₋₆-alkyl heterocycloalkyl”.

The term “C₁₋₆-alkyl sulfonyl” as used herein refers to a C₁₋₆ alkyl substituted by a sulfonyl as defined here above.

The term “sulfonyloxy” as used herein refers to a group of formula “—OSO₂—R^{k}” wherein R^{k} is defined as here above for sulfonyl group.

The term “C₁₋₆-alkyl sulfonyloxy” as used herein refers to a C₁₋₆ alkyl substituted by a sulfonyloxy as defined here above.

The term “aminosulfonyl” as used herein refers to a group of formula —SO₂—NR^{c}R^{d} wherein R^{c} and R^{d} are as defined here above for the amino group.

The term “C₁₋₆-alkyl aminosulfonyl” as used herein refers to a C₁₋₆ alkyl substituted by an aminosulfonyl as defined here above.

The term “sulfinyl” as used herein refers to a group “—S(O)—R^{k}” wherein R^{k} is as defined here above for sulfonyl group.

The term “C₁₋₆-alkyl sulfinyl” as used herein refers to a C₁₋₆ alkyl substituted by a sulfinyl as defined here above.

The term “sulfanyl” as used herein refers to a group of formula —S—R^{k} where R^{k} is as defined here above for sulfonyl group.

The term “C₁₋₆-alkyl sulfanyl” as used herein refers to a C₁₋₆ alkyl substituted by a sulfanyl as defined here above.

The term “sulfonylamino” as used herein refers to a group —NR^{c}SO₂—R^{k} wherein R^{k} is defined as here above for sulfonyl group and Rc is defined as here above for amino group.

The term “C₁₋₆-alkyl sulfonylamino” as used herein refers to a C₁₋₆ alkyl substituted by a sulfonylamino as defined here above.

The term “phosphonate” as used herein refers to a group of formula —P(O)—(OR^{m})₂ wherein R^{m} is an alkyl group as defined herein.

The term “C₁₋₆-alkyl phosphonate” refers to a C₁₋₆ alkyl group substituted by a “phosphonate” as described here above.

Unless otherwise constrained by the definition of the individual substituents, all the above set out groups may be “substituted” or unsubstituted”.

“Substituted or unsubstituted” as used herein, unless otherwise constrained by the definition of the individual substituents, shall mean that the above set out groups, like “C₁₋₆ alkyl”, “C₂₋₆ alkenyl”, “C₂₋₆ alkynyl”, “aryl” and “heteroaryl” etc. . . . may optionally be substituted with from 1 to 5 substituents selected from the group consisting of “C₁₋₆ alkyl”, “C₂₋₆ alkenyl”, “C₂₋₆ alkynyl”, “cycloalkyl”, “heterocycloalkyl”, “C₁₋₆-alkyl heterocycloalkyl”, “amino”, “halogen”, “hydroxy” and the like.

In one embodiment according to the present invention, A represents a group of formula —NR²R³ wherein R² and R³ are independently substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, substituted or unsubstituted heterocycloalkyl, substituted or unsubstituted C₁₋₆-alkyl aryl, substituted or unsubstituted C₁₋₆-alkyl heteroaryl, substituted or unsubstituted C₁₋₆-alkyl cycloalkyl or substituted or unsubstituted C₁₋₆-alkyl heterocycloalkyl groups; or A is a 3 to 8 membered substituted or unsubstituted heterocycloalkyl linked to the cyclobutyl group via a nitrogen atom.

In another embodiment according to the present invention, A is a group —NR²R³ wherein R² and R³ are independently substituted or unsubstituted C₁₋₆ alkyl; or A is a 3 to 8 membered substituted or unsubstituted heterocycloalkyl linked to the cyclobutyl group via a nitrogen atom.

In a particular embodiment according to the present invention, A is a 3 to 8 membered heterocycloalkyl linked to the cyclobutyl group via a nitrogen atom.

In another particular embodiment according to the present invention, A represents a 3 to 8 membered heterocycloalkyl selected from the groups comprising or consisting of substituted or unsubstituted piperidin-1-yl, substituted or unsubstituted morpholin-4-yl, substituted or unsubstituted pyrrolidin-1-yl, substituted or unsubstituted piperazin-1-yl, substituted or unsubstituted azepan-1-yl or substituted or unsubstituted thiomorpholin-4-yl.

In one particular embodiment according to the present invention, A is selected from substituted or unsubstituted piperidin-1-yl, and substituted or unsubstituted pyrrolidin-1-yl.

In another particular embodiment, A is piperidin-1-yl, 2-methylpyrrolidin-1-yl, (2R)-2-methylpyrrolidin-1-yl or (2S)-2-methylpyrrolidin-1-yl.

Generally, A¹ may be CH, C—F, C—Cl, C—O—CH₃ or N. In a particular embodiment, A¹ is CH, C—F or C—Cl. In another particular embodiment, A¹ is CH.

In one embodiment according to the present invention, B represents a group of formula —NR⁴R⁵ wherein R⁴ and R⁵ are independently hydrogen, substituted or unsubstituted C₁₋₆ alkyl, substituted or unsubstituted aryl or substituted or unsubstituted heteroaryl, provided that at least one of R⁴ and R⁵ is different from hydrogen. Typical examples of such —NR⁴R⁵ groups are (3-chloropyridin-4-yl)amino, (4-aminopyridin-3-yl)amino and (2,2,2-trifluoroethyl)amino.

In another embodiment according to the present invention, B is a 3 to 8 membered substituted or unsubstituted heterocycloalkyl linked to the carbonyl or thiocarbonyl group via a nitrogen atom.

In another particular embodiment according to the present invention, B represents a 3 to 8 membered heterocycloalkyl selected from the groups comprising or consisting of substituted or unsubstituted piperidin-1-yl, substituted or unsubstituted morpholin-4-yl, substituted or unsubstituted pyrrolidin-1-yl, substituted or unsubstituted piperazin-1-yl, substituted or unsubstituted thiomorpholin-4-yl, substituted or unsubstituted azetidin-1-yl, substituted or unsubstituted imidazolidin-1-yl, substituted or unsubstituted isoxazolidin-2-yl, substituted or unsubstituted 1,3-thiazolidin-3-yl, substituted or unsubstituted pyrazolidin-1-yl and substituted or unsubstituted 1,4-oxazepan-4-yl.

Typical examples of B according to the invention include morpholin-4-yl, 4,4-difluoropiperidin-1-yl, piperidin-1-yl, 4-isopropylpiperazin1-yl, 4-hydroxypiperazin-1-yl, thiomorpholin-4-yl, pyrrolidin-1-yl, 1,1-dioxidothiomorpholin-4-yl, 3,3-difluoropyrrolidin-1-yl, 3-hydroxyazetidin-1-yl, 4-acetylpiperazin-1-yl, 4-oxoimidazolidin-1-yl, 2-(methoxymethyl)pyrrolidin-1-yl, 4-hydroxyisoxazolidin-2-yl, 1,3-thiazolidin-3-yl, 3-oxopyrazolidin-1-yl, 4-hydroxyoxazolidin-2-yl, 1,4-oxazepan-4-yl, 4-carbamoylpiperidin-1-yl and 4-oxopiperidin-1-yl.

In one particular embodiment according to the present invention, B is selected from substituted or unsubstituted piperidin-1-yl, substituted or unsubstituted morpholin-4-yl, substituted or unsubstituted pyrrolidin-1-yl or substituted or unsubstituted 1,4-oxazepan-4-yl.

In another particular embodiment according to the present invention, B is selected from substituted or unsubstituted piperidin-1-yl and substituted or unsubstituted morpholin-4-yl.

In a particular embodiment according of the present invention, X is O. In another particular embodiment according of the present invention, X is S.

In a particular embodiment according to the invention, Y is O. In another particular embodiment according of the present invention, Y is S.

In one embodiment according to the present invention, R¹ is hydrogen, C₁₋₆ alkoxy or halogen.

In another embodiment according to the present invention, R¹ is hydrogen, methoxy, chlorine or fluorine.

In a particular embodiment according to the present invention, R¹ is hydrogen. In a particular embodiment, the present invention relates to compounds of formula (I), geometrical isomers, enantiomers, diastereoisomers, pharmaceutically acceptable salts and all possible mixtures thereof,

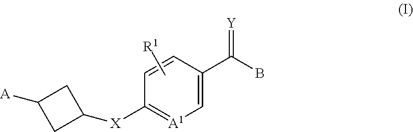

wherein

A is a 3 to 8 membered substituted or unsubstituted heterocycloalkyl linked to the cyclobutyl group via a nitrogen atom;

A¹ is CH, C—Cl, C—F or C—O—CH₃;

Y is O;

B is a 3 to 8 membered substituted or unsubstituted heterocycloalkyl linked to the carbonyl group via a nitrogen atom; or B is a group of formula —NR⁴R⁵ wherein R⁴ and R⁵ are independently hydrogen, substituted or unsubstituted C₁₋₆ alkyl, substituted or unsubstituted aryl or substituted or unsubstituted heteroaryl, provided that at least one of R⁴ and R⁵ is different from hydrogen.

X is O and

R¹ is hydrogen, chlorine, fluorine or methoxy.

In another particular embodiment, the present invention relates to compounds of formula (I), geometrical isomers, enantiomers, diastereoisomers, pharmaceutically acceptable salts and all possible mixtures thereof,

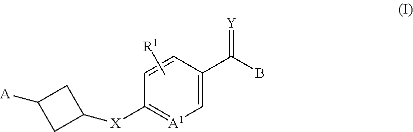

wherein

A is a 3 to 8 membered heterocycloalkyl selected from the groups comprising or consisting of substituted or unsubstituted piperidin-1-yl, substituted or unsubstituted morpholin-4-yl, substituted or unsubstituted pyrrolidin-1-yl, substituted or unsubstituted piperazin-1-yl, substituted or unsubstituted azepan-1-yl or substituted or unsubstituted thiomorpholin-4-yl;

A¹ is CH, C—Cl, C—F or C—O—CH₃;

Y is O;

B is a 3 to 8 membered heterocycloalkyl selected from the groups comprising or consisting of substituted or unsubstituted piperidin-1-yl, substituted or unsubstituted morpholin-4-yl, substituted or unsubstituted pyrrolidin-1-yl, substituted or unsubstituted piperazin-1-yl, substituted or unsubstituted thiomorpholin-4-yl, substituted or unsubstituted azetidin-1-yl, substituted or unsubstituted imidazolidin-1-yl, substituted or unsubstituted isoxazolidin-2-yl, substituted or unsubstituted 1,3-thiazolidin-3-yl, substituted or unsubstituted pyrazolidin-1-yl and substituted or unsubstituted 1,4-oxazepan-4-yl.X is O; and

R¹ is hydrogen, chlorine, fluorine or methoxy.

In another particular embodiment, the present invention relates to compounds of formula (I), geometrical isomers, enantiomers, diastereoisomers, pharmaceutically acceptable salts and all possible mixtures thereof,

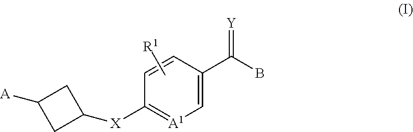

wherein

A is a 3 to 8 membered heterocycloalkyl selected from the groups comprising or consisting of substituted or unsubstituted piperidin-1-yl or substituted or unsubstituted pyrrolidin-1-yl;

A¹ is CH;

Y is O;

B is a 3 to 8 membered heterocycloalkyl selected from the groups comprising or consisting of substituted or unsubstituted piperidin-1-yl or substituted or unsubstituted morpholin-4-yl;

X is O; and

R¹ is hydrogen.

In one aspect, the present invention relates to compounds of formula (Ia), geometrical isomers, enantiomers, diastereoisomers, pharmaceutically acceptable salts and all possible mixtures thereof,

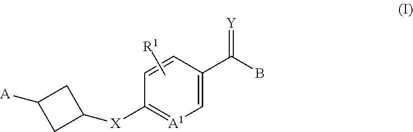

wherein A, A¹, B, X, Y and R¹ are as herein defined.

Embodiments described hereinabove for A, A¹, X, Y, B and R¹ in compounds of formula (I) also apply to A, A¹, X, Y, B and R¹ in compounds of formula (Ia).

In another aspect, the present invention relates to compounds of formula (Ib), geometrical isomers, enantiomers, diastereoisomers, pharmaceutically acceptable salts and all possible mixtures thereof,

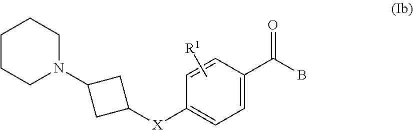

wherein X and R¹ are as herein defined; and

B is a substituted or unsubstituted 3-8 membered heterocycloalkyl which is linked to the carbonyl via an amino nitrogen.

Embodiments described hereinabove for R¹ and X in compounds of formula (I) also apply to R¹ and X in compounds of formula (Ib).

In another aspect, the present invention relates to compounds of formula (Ic), geometrical isomers, enantiomers, diastereoisomers, pharmaceutically acceptable salts and all possible mixtures thereof,

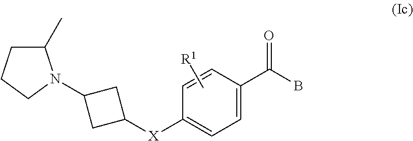

wherein X and R¹ are as herein defined; and

B is a substituted or unsubstituted 3-8 membered heterocycloalkyl which is linked to the carbonyl via an amino nitrogen.

Embodiments described hereinabove for R¹ and X in compounds of formula (I) also apply to R¹ and X in compounds of formula (Ic).

According to a specific embodiment of compounds of formula (I), (Ib) and (Ic), the A and X groups attached to the cyclobutyl in the A-cyclobutyl-X moiety are in trans configuration.

Examples of compounds according to the present invention are:
4-{4-[(trans-3-piperidin-1-ylcyclobutyl)oxy]benzoyl}morpholine;
4,4-difluoro-1-{4-[(trans-3-piperidin-1-ylcyclobutyl)oxy]benzoyl}piperidine;
1-{trans-3-[4-(piperidin-1-ylcarbonyl)phenoxy]cyclobutyl}piperidine;
1-isopropyl-4-{4-[(trans-3-piperidin-1-ylcyclobutyl)oxy]benzoyl}piperazine;
1-{4-[(trans-3-piperidin-1-ylcyclobutyl)oxy]benzoyl}piperidin-4-ol;
4-{4-[(trans-3-piperidin-1 -ylcyclobutyl)oxy]benzoyl}thiomorpholine;
1-{trans-3-[4-(pyrrolidin-1-ylcarbonyl)phenoxy]cyclobutyl}piperidine;
4-{4-[(trans-3-piperidin-1-ylcyclobutyl)oxy]benzoyl}thiomorpholine 1,1-dioxide;
1-(trans-3-{4-[(3,3-difluoropyrrolidin-1-yl)carbonyl]phenoxy}cyclobutyl)piperidine;
1-acetyl-4-{4-[(trans-3-piperidin-1-ylcyclobutyl)oxy]benzoyl}piperazine; and
1-{4-[(trans-3-piperidin-1-ylcyclobutyl)oxy]benzoyl}azetidin-3-ol;
4-[(trans-3-piperidin-1-ylcyclobutyl)oxy]-N-(2,2,2-trifluoroethyl)benzamide;
4-[4-({trans-3-[(2R)-2-methylpyrrolidin-1-yl]cyclobutyl}oxy)benzoyl]morpholine;4-[4-({trans-3-[(2S)-2-methylpyrrolidin-1-yl]cyclobutyl}oxy)benzoyl]morpholine;
4-(4-{[trans-3-(2-methylpyrrolidin-1-yl)cyclobutyl]oxy}benzoyl)morpholine;
4-{[(4-({trans-3-[(2S)-2-methylpyrrolidin-1-yl]cyclobutyl}oxy)phenyl]carbonothioyl}morpholine;
4-{3-chloro-4-[(trans-3-piperidin-1-ylcyclobutyl)oxy]benzoyl}morpholine;
1-{2-chloro-4-[(trans-3-piperidin-1-ylcyclobutyl)oxy]benzoyl}imidazolidin-4-one;
4-{2-fluoro-4-[(trans-3-piperidin-1-ylcyclobutyl)oxy]benzoyl}morpholine;
1-[trans-3-(3-chloro-4-{[2-(methoxymethyl)pyrrolidin-1-yl]carbonyl}phenoxy)cyclobutyl]piperidine;
2-{3-chloro-4-[(trans-3-piperidin-1-ylcyclobutyl)oxy]benzoyl}isoxazolidin-4-ol;
1-{trans-3-[3-chloro-4-(1,3-thiazolidin-3-ylcarbonyl)phenoxy]cyclobutyl}piperidine;
1-{4-[(trans-3-piperidin-1-ylcyclobutyl)oxy]benzoyl}pyrazolidin-3-one;
2-{4-[(trans-3-piperidin-1-ylcyclobutyl)oxy]benzoyl}isoxazolidin-4-ol;
4-{3-chloro-4-[(trans-3-piperidin-1-ylcyclobutyl)oxy]benzoyl}-1,4-oxazepane;
1-{4-[(trans-3-piperidin-1-ylcyclobutyl)oxy]benzoyl}piperidine-4-carboxamide;
1-{trans-3-[3-chloro-4-(pyrrolidin-1-ylcarbonyl)phenoxy]cyclobutyl}piperidine;
4-{2-fluoro-4-[(trans-3-piperidin-1-ylcyclobutyl)oxy]benzoyl}-1,4-oxazepane;
1-[trans-3-(4-{[2-(methoxymethyl)pyrrolidin-1-yl]carbonyl}phenoxy)cyclobutyl]piperidine;
4-{2-chloro-4-[(trans-3-piperidin-1-ylcyclobutyl)oxy]benzoyl}-1,4-oxazepane;
2-{2-chloro-4-[(trans-3-piperidin-1-ylcyclobutyl)oxy]benzoyl}isoxazolidin-4-ol;
1-{3-fluoro-4-[(trans-3-piperidin-1-ylcyclobutyl)oxy]benzoyl}piperidin-4-one;
1-{trans-3-[4-(1,3-thiazolidin-3-ylcarbonyl)phenoxy]cyclobutyl}piperidine;
1-{2-chloro-4-[(trans-3-piperidin-1-ylcyclobutyl)oxy]benzoyl}piperidine-4-carboxamide;
1-{4-[(trans-3-piperidin-1-ylcyclobutyl)oxy]benzoyl}imidazolidin-4-one;
1-[trans-3-(3-fluoro-4-{[2-(methoxymethyl)pyrrolidin-1-yl]carbonyl}phenoxy)cyclobutyl]piperidine;
1-{3-methoxy-4-{trans-3-piperidin-1-ylcyclobutyl)oxy}benzoyl}imidazolidin-4-one;
4-{4-[(trans-3-piperidin-1-ylcyclobutyl)oxy]benzoyl}-1,4-oxazepane;
4-{2-chloro-4-[(trans-3-piperidin-1-ylcyclobutyl)oxy]benzoyl}morpholine;
1-{trans-3-[3-fluoro-4-(1,3-thiazolidin-3-ylcarbonyl)phenoxy]cyclobutyl}piperidine;
4-{4-[(trans-3-piperidin-1-ylcyclobutyl)thio]benzoyl}morpholine; and
1-{4-[(trans-3-piperidin-1-ylcyclobutyl)thio]benzoyl}piperidin-4-one.

The compounds of the present invention are histamine H₃-receptor ligands. In one embodiment they are histamine H₃-receptor antagonists; in another embodiment they are histamine H₃-receptor inverse agonists.

In one embodiment, compounds of the present invention have particularly favorable drug properties, i.e. they have a good affinity to the H₃-receptor while having a low affinity towards other receptors or proteins; they have favorable pharmacokinetics and pharmacodynamics while having few side effects, e.g. toxicity such as cardiotoxicity. One of many methods known to determine the cardiovascular risk of drug compounds is to assess the binding of a test compound to hERG channels.

Compounds of the present invention display a particular low affinity on hERG channels.

Moreover, preferred compounds according to the present invention exhibit good brain H₃ receptor occupancy.

The “pharmaceutically acceptable salts” according to the invention include therapeutically active, non-toxic acid salt forms which the compounds of formula (I) are able to form.

The acid addition salt form of a compound of formula (I) that occurs in its free form as a base can be obtained by treating the free base with an appropriate acid such as an inorganic acid, for example, a hydrochloric, hydrobromic, sulfuric, nitric, phosphoric and the like; or an organic acid, such as, for example, acetic, trifluoroacetic, hydroxyacetic, propanoic, lactic, pyruvic, malonic, succinic, maleic, fumaric, malic, tartaric, citric, methanesulfonic, ethanesulfonic, benzenesulfonic, p-toluenesulfonic, cyclamic, salicylic, p-aminosalicylic, palmoic, and the like.

Conversely said salt forms can be converted into the free forms by treatment with an appropriate base.

Compounds of the formula (I) and their salts can be in the form of a solvate, which is included within the scope of the present invention. Such solvates include for example hydrates, alcoholates and the like.

Some of the compounds of formula (I) and some of their intermediates have at least one stereogenic center in their structure. This stereogenic center may be present in a R or a S configuration, said R and S notation is used in correspondence with the rules described in Pure Appl. Chem. 1976, 45, 11-30.

The invention also relates to all stereoisomeric forms such as enantiomeric and diastereomeric forms of the compounds of formula (I) or mixtures thereof (including all possible mixtures of stereoisomers).

With respect to the present invention reference to a compound or compounds is intended to encompass that compound in each of its possible isomeric forms and mixtures thereof, unless the particular isomeric form is referred to specifically.

The expression “enantiomerically pure” as used herein refers to compounds which have an enantiomeric excess (ee) greater 95%.

Compounds according to the present invention may exist in different polymorphic forms. Although not explicitly indicated in the above formula, such forms are included within the scope of the present invention.

The invention also includes within its scope pro-drug forms of the compounds of formula (I) and its various sub-scopes and sub-groups.

The term “prodrug” as used herein includes compound forms which are rapidly transformed in vivo to the parent compound according to the invention, for example, by hydrolysis in blood. Prodrugs are compounds bearing groups which are removed by biotransformation prior to exhibiting their pharmacological action. Such groups include moieties which are readily cleaved in vivo from the compound bearing it, which compound after cleavage remains or becomes pharmacologically active. Metabolically cleavable groups form a class of groups well known to practitioners of the art. They include, but are not limited to such groups as alkanoyl (i.e. acetyl, propionyl, butyryl, and the like), unsubstituted and substituted carbocyclic aroyl (such as benzoyl, substituted benzoyl and 1- and 2-naphthoyl), alkoxycarbonyl (such as ethoxycarbonyl), trialklysilyl (such as trimethyl- and triethylsilyl), monoesters formed with dicarboxylic acids (such as succinyl), phosphate, sulfate, sulfonate, sulfonyl, sulfinyl and the like. The compounds bearing the metabolically cleavable groups have the advantage that they may exhibit improved bioavailability as a result of enhanced solubility and/or rate of absorption conferred upon the parent compound by virtue of the presence of the metabolically cleavable group. T. Higuchi and V. Stella, “Pro-drugs as Novel Delivery System”, Vol. 14 of the A.C.S. Symposium Series; “Bioreversible Carriers in Drug Design”, ed. Edward B. Roche, American Pharmaceutical Association and Pergamon Press, 1987.

Compounds of formula (I) according to the invention may be prepared according to conventional methods known to the person skilled in the art of synthetic organic chemistry.

A. According to one embodiment, compounds of formula (I) wherein Y is O, may be prepared by aminocarbonylation of a compound of formula (II) according to the equation

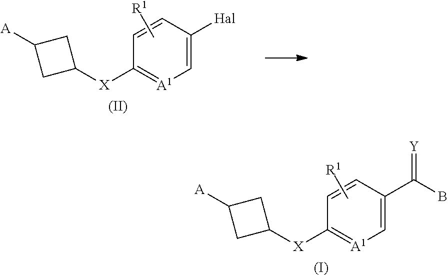

wherein Hal is iodine or bromine, Y is O, A, A¹, R¹, X and B having the same definition as in the general formula above for compounds of formula (I).

This reaction may be carried out in the presence of a carbon monoxide source such as molybdenum hexacarbonyl, a suitable catalyst such as palladium acetate, and a base such as 1,8-diazabicyclo[5.4.0]undec-7-ene in a solvent such as dry tetrahydrofuran, and under microwave irradiation according to the method described by Letavic M. et al. in Tetrahedron Lett., 2007, 48, 2339-2343, or according to any other method known to the man skilled in the art.

Alternatively, compounds of formula (I) may be prepared from compounds of formula (II) by lithium-halogen exchange in the presence of butyllithium or other lithium-releasing agents known to the man skilled in the art, followed by treatment with carbon dioxide or ethyl chloroformate or any other suitable reagent known to the man skilled in the art. The resulting carboxylic acids or esters are then easily converted to the desired amides by any method know to the man skilled in the art.

A.1. Some compounds of formula (II) wherein A¹ is CH or C-halogen may be prepared by reaction of a compound of formula (IV) with a compound of formula (III) according to the equation

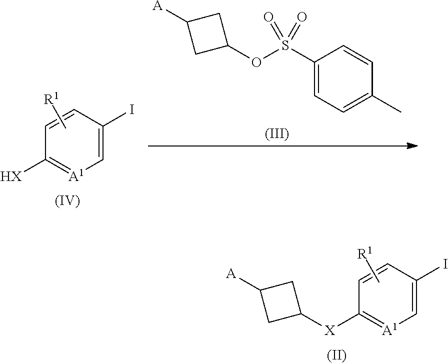

wherein A¹ is CH or C-halogen, A, R¹ and X having the same definition as in the general formula above for compounds of formula (I).

This reaction may be carried out in the presence of a base, for example sodium hydride, in a solvent, for example N,N-dimethylacetamide, under an inert atmosphere, at a temperature ranging from 50° C. to 80° C., or in any other conditions that the man skilled in the art will deem appropriate, and according to conventional methods known to him.

Compounds of formula (IV) may be commercially available or prepared according to any conventional methods known to the man skilled in the art.

Compounds of formula (III) may be prepared by reaction of a compound of formula (V) with p-toluenesulfonyl chloride according to the equation

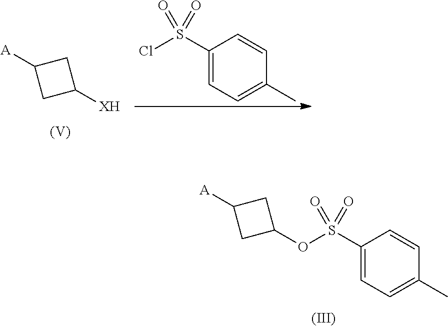

wherein X is O and A has the same definition as described above for compounds of formula (I).

This reaction may be carried out using a base such as triethylamine or N-methylimidazole, in a solvent such as dichloromethane, at a temperature ranging from 0° C. to 25° C., under an inert atmosphere (argon or nitrogen), or according to any conventional method known by the man skilled in the art.

Compounds of formula (V) wherein X is O may be prepared from compounds of formula (VI), according to the equation

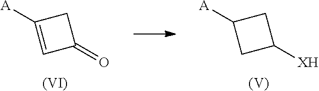

wherein X is O and A has the same definition as described above for compounds of formula (I).

This reaction may be carried out using a reductive agent such as sodium borohydride, in a protic solvent such as ethanol, at a temperature ranging from 0° C. to 60° C., or according to any conventional method known by the man skilled in the art.

Compounds of formula (V) wherein X is S may be prepared from compound of formula (III) according to the equation:

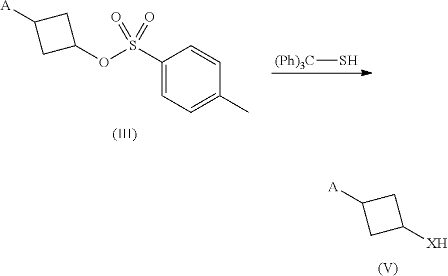

wherein X is S and A has the same definition as described above for compounds of formula I.

This reaction may be carried out according to the method described by Oh, C. -H. and Sho, J. -H. in Eur. J. Med. Chem. 2006, 41, 50-55, i.e., using triphenylmethylthiol in the presence of a base (e.g., sodium hydride) and an inert solvent (e.g., dimethylformamide), at a temperature ranging from 0° C. to 100° C., under an inert atmosphere (argon or nitrogen), followed by deprotection of the triphenylmethyl group using a trifluoroacetic acid/triethylsilane reductive system. Alternatively, this reaction scheme may be performed according to any other conventional method known by the man skilled in the art.

Compounds of formula (VI) may be commercially available or prepared from cyclobutane-1,3-dione (VII) by reaction with an amine of formula AH, according to the equation

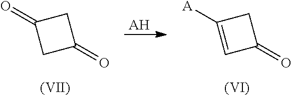

wherein A has the same definition as described above for compounds of formula I.

This reaction may be carried out in a solvent such as dioxane, at a temperature ranging from 0° C. to 60° C., or according to any conventional method known by the man skilled in the art. Cyclobutan-1,3-dione is commercially available or may be prepared according to any conventional method known to the person skilled in the art.

A.2. Some compounds of formula (II) wherein A¹ is N may be prepared by reaction of a compound of formula (VIII) with a compound of formula (V) according to the equation

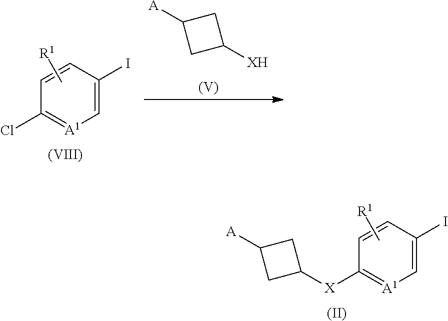

wherein A¹ is N, A, R¹ and X having the same definition as in the general formula above for compounds of formula (I). This reaction may be carried out in the presence of a base such as potassium tert-butylate, in a solvent such as N,N-dimethylacetamide, between 25 and 120° C., or according to any other method known to the person skilled in the art.

B. According to another embodiment, some compounds of formula (I) where Y is S may be prepared by thionation of the parent carbonyl compounds of formula (I) wherein Y is O. This reaction may be performed with Lawesson's reagent in tetrahydrofuran or according to any other methods known to the man skilled in the art.

Examples of synthetic intermediates used for the synthesis of compounds of formula (I) according to the present invention are:
N-cyclohexylcyclohexanaminium 3-oxocyclobut-1-en-1-olate;
3-piperidin-1-ylcyclobut-2-en-1-one;
cis-3-piperidin-1-ylcyclobutanol;
cis-3-piperidin-1-ylcyclobutyl 4-methylbenzenesulfonate;
1-[trans-3-(4-iodophenoxy)cyclobutyl]piperidine;
3-(2-methylpyrrolidin-1-yl)cyclobut-2-en-1-one;
cis-3-(2-methylpyrrolidin-1-yl)cyclobutanol;
cis-3-(2-methylpyrrolidin-1-yl)cyclobutyl 4-methylbenzenesulfonate;
1-[trans-3-(4-iodophenoxy)cyclobutyl]-2-methylpyrrolidine;
(2S)-1-[trans-3-(4-iodophenoxy)cyclobutyl]-2-methylpyrrolidine;
(2R)-1-[trans-3-(4-iodophenoxy)cyclobutyl]-2-methylpyrrolidine;
cis-3-(piperidin-1-yl)cyclobutyl 4-bromobenzenesulfonate;
4-{[trans-3-(piperidin-1-yl)cyclobutyl]sulfanyl}benzoic acid;
1-[trans-3-(4-bromophenoxy)cyclobutyl]piperidine;
1-[trans-3-(4-bromo-2-fluorophenoxy)cyclobutyl]piperidine;
1-[trans-3-(4-bromo-2-methoxyphenoxy)cyclobutyl]piperidine;
1-[trans-3-(4-bromo-2-chlorophenoxy)cyclobutyl]piperidine;
1-[trans-3-(4-bromo-3-fluorophenoxy)cyclobutyl]piperidine;
1-[trans-3-(4-bromo-3-chlorophenoxy)cyclobutyl]piperidine;
4-{[trans-3-(piperidin-1-yl)cyclobutyl]oxy}benzoic acid;
3-fluoro-4-{[trans-3-(piperidin-1-yl)cyclobutyl]oxy}benzoic acid;
3-methoxy-4-{[trans-3-(piperidin-1-yl)cyclobutyl]oxy}benzoic acid;
3-chloro-4-{[trans-3-(piperidin-1-yl)cyclobutyl]oxy}benzoic acid;
2-fluoro-4-{[trans-3-(piperidin-1-yl)cyclobutyl]oxy}benzoic acid; and
2-chloro-4-{[trans-3-(piperidin-1-yl)cyclobutyl]oxy}benzoic acid.

It has now been found that compounds of formula (I) according to the present invention and their pharmaceutically acceptable salts are useful in a variety of medical disorders.

For example, the compounds according to the invention are useful for the treatment and prevention of diseases or pathological conditions of the central nervous system including mild-cognitive impairments, Alzheimer's disease, learning and memory disorders, cognitive disorders, attention deficit disorder, attention-deficit hyperactivity disorder, Parkinson's disease, schizophrenia, dementia, depression, epilepsy, seizures, convulsions, sleep/wake and arousal/vigilance disorders such as hypersomnia and narcolepsy, pain and/or obesity.

Furthermore, compounds according to the invention alone or in combination with an antiepileptic drug (AED) may be useful in the treatment of epilepsy, seizure or convulsions. It is known from literature that the combination of H₃-receptor ligands with an AED may produce additive synergistic effects on efficacy with reduced side-effects such as decreased vigilance, sedation or cognitive problems.

Furthermore, compounds of general formula (I) alone or in combination with a histamine H₁ antagonist may also be used for the treatment of upper airway allergic disorders.

In a particular embodiment of the present invention, compounds of general formula (I), alone or in combination with muscarinic receptor ligands and particularly with a muscarinic M₂ antagonist, may be useful for the treatment of cognitive disorders, Alzheimer's disease, and attention-deficit hyperactivity disorder.

Particularly, compounds of general formula (I) displaying NO-donor properties, alone or in combination with a nitric oxide (NO) releasing agent may be useful in the treatment of cognitive dysfunctions.

Compounds of general formula (I) may also be used in the treatment and prevention of multiple sclerosis (MS).

Usually, compounds of general formula (I) may be used in the treatment and prevention of all types of cognitive-related disorders.

In one embodiment, compounds of general formula (I) may be used for the treatment and prevention of cognitive dysfunctions in diseases such as mild cognitive impairment, dementia, Alzheimer's disease, Parkinson's disease, Down's syndrome as well as for the treatment of attention-deficit hyperactivity disorder.

In another embodiment, compounds of general formula (I) may also be used for the treatment and prevention of psychotic disorders, such as schizophrenia; or for the treatment of eating disorders, such as obesity; or for the treatment of inflammation and pain disorders; or for the treatment of anxiety, stress and depression; or for the treatment of cardiovascular disorders, for example, myocardial infarction; or for the treatment and prevention of multiple sclerosis (MS).

Pain disorders include neuropathic pain, such as associated with diabetic neuropathy, post-herpetic neuralgia; trigeminal neuralgia, posttraumatic peripheral neuropathy, phantom limb pain, with cancer and neuropathies induced by treatment with antineoplastic agents, pain due to nerve damage associated with demyelinating disease such as multiple sclerosis, neuropathy associated with HIV, post-operative pain; corneal pain, obstetrics pain (pain relief during delivery or after caesarean section), visceral pain, inflammatory pain such as associated to rheumatoid arthritis; low-back pain/sciatica; carpal tunnel syndrome, allodynic pain such as fibromyalgia; chronic pain associated with Complex Regional Pain Syndrome (CRPS) and chronic muscle pain such as, yet not limited to, that associated with back spasm.

In a particular embodiment, compounds of formula (I) may be used for the treatment and prevention neuropathic pain.

In one embodiment, compounds of formula (I) according to the present invention may be used as a medicament.

In another embodiment, compounds of formula (I) according to the present invention may be used for the treatment or prevention of mild-cognitive impairement, Alzheimer's disease, learning and memory disorders, attention-deficit hyperactivity disorder, Parkinson's disease, schizophrenia, dementia, depression, epilepsy, seizures, convulsions, sleep/wake disorders, cognitive dysfunctions, narcolepsy, hypersomnia, obesity, upper airway allergic disorders, Down's syndrome, anxiety, stress, cardiovascular disorders, inflammation, pain disorders, particularly neuropathic pain, or multiple sclerosis.

In a particular embodiment, compounds of formula (I) according to the present invention may be used for the treatment of mild cognitive impairment, dementia, Alzheimer's disease, Parkinson's disease, Down's syndrome as well as for the treatment of attention-deficit hyperactivity disorder.

In a further embodiment, the present invention concerns the use of a compound of formula (I) or a pharmaceutically acceptable salt thereof or of a pharmaceutical composition comprising an effective amount of said compound for the manufacture of a medicament for the treatment and prevention of mild-cognitive impairement, Alzheimer's disease, learning and memory disorders, attention-deficit hyperactivity disorder, Parkinson's disease, schizophrenia, dementia, depression, epilepsy, seizures, convulsions, sleep/wake disorders, cognitive dysfunctions, narcolepsy, hypersomnia, obesity, upper airway allergic disorders, Down's syndrome, anxiety, stress, cardiovascular disorders, inflammation, pain disorders, particularly neuropathic pain, or multiple sclerosis.

In another embodiment, the present invention concerns the use of a compound of formula (I) or a pharmaceutically acceptable salt thereof or a pharmaceutical composition comprising an effective amount of said compound for the manufacture of a medicament for the treatment of cognitive dysfunctions in diseases such as mild cognitive impairment, dementia, Alzheimer's disease, Parkinson's disease, Down's syndrome as well as for the treatment of attention-deficit hyperactivity disorder.

The methods of the invention comprise administration to a mammal (preferably human) suffering from above mentioned conditions or disorders, of a compound according to the invention in an amount sufficient to alleviate or prevent the disorder or condition.

The compound is conveniently administered in any suitable unit dosage form, including but not limited to one containing 3 to 3000 mg of active ingredient per unit dosage form.

The term “treatment” as used herein includes curative treatment and prophylactic treatment.

By “curative” is meant efficacy in treating a current symptomatic episode of a disorder or condition.

By “prophylactic” is meant prevention of the occurrence or recurrence of a disorder or condition.

The term “cognitive disorders” as used herein refers to disturbances of cognition, which encompasses perception, learning and reasoning or in other terms the physiological (mental/neuronal) process of selectively acquiring, storing, and recalling information.

The term “attention-deficit hyperactivity disorder” (ADHD) as used herein refers to a problem with inattentiveness, over-activity, impulsivity, or a combination of these. For these problems to be diagnosed as ADHD, they must be out of the normal range for the child's age and development. The term “attention-deficit disorder” (ADD) is also commonly used for the same disorder.

The term “Alzheimer's disease” (AD) as used herein refers to a progressive, neurodegenerative disease characterized in the brain by abnormal clumps (amyloid plaques) and tangled bundles of fibers (neurofibrillary tangles) composed of misplaced proteins. Age is the most important risk factor for AD; the number of people with the disease doubles every 5 years beyond age 65. Three genes have been discovered that cause early onset (familial) AD. Other genetic mutations that cause excessive accumulation of amyloid protein are associated with age-related (sporadic) AD. Symptoms of AD include memory loss, language deterioration, impaired ability to mentally manipulate visual information, poor judgment, confusion, restlessness, and mood swings. Eventually AD destroys cognition, personality, and the ability to function. The early symptoms of AD, which include forgetfulness and loss of concentration, are often missed because they resemble natural signs of aging.

The term “Parkinson's disease” (PD) as used herein refers to a group of conditions called motor system disorders, which are the result of the loss of dopamine-producing brain cells. The four primary symptoms of PD are tremor, or trembling in hands, arms, legs, jaw, and face; rigidity, or stiffness of the limbs and trunk; bradykinesia, or slowness of movement; and postural instability, or impaired balance and coordination. As these symptoms become more pronounced, patients may have difficulty walking, talking, or completing other simple tasks. PD usually affects people over the age of 50. Early symptoms of PD are subtle and occur gradually. In some people the disease progresses more quickly than in others. As the disease progresses, the shaking, or tremor, which affects the majority of PD patients may begin to interfere with daily activities. Other symptoms may include depression and other emotional changes; difficulty in swallowing, chewing, and speaking; urinary problems or constipation; skin problems; and sleep disruptions.

The term “Down's syndrome” as used herein refers to a chromosome abnormality, usually due to an extra copy of the 21st chromosome. This syndrome, usually but not always results in mental retardation and other conditions. The term “mental retardation” refers to a below-average general intellectual function with associated deficits in adaptive behavior that occurs before age 18.

The term “mild-cognitive impairment” as used herein refers to a transitional stage of cognitive impairment between normal aging and early Alzheimer's disease. It refers particularly to a clinical state of individuals who are memory impaired but are otherwise functioning well and do not meet clinical criteria for dementia.

The term “obesity” as used herein refers to a body mass index (BMI) which is greater than 30 kg/m².

The term “dementia” as used herein refers to a group of symptoms involving progressive impairment of brain function. American Geriatrics Society refers to dementia as a condition of declining mental abilities, especially memory. The person will have problems doing things he or she used to be able to do, like keep the check book, drive a car safely, or plan a meal. He or she will often have problems finding the right words and may become confused when given too many things to do at once. The person with dementia may also change in personality, becoming aggressive, paranoid, or depressed.

The term “schizophrenia” as used herein refers to a group of psychotic disorders characterized by disturbances in thought, perception, attention, affect, behavior, and communication that last longer than 6 months. It is a disease that makes it difficult for a person to tell the difference between real and unreal experiences, to think logically, to have normal emotional responses to others, and to behave normally in social situations.

The term “anxiety” as used herein refers to a feeling of apprehension or fear. Anxiety is often accompanied by physical symptoms, including twitching or trembling, muscle tension, headaches, sweating, dry mouth, difficulty swallowing and/or abdominal pain.

The term “narcolepsy” as used herein refers to a sleep disorder associated with uncontrollable sleepiness and frequent daytime sleeping.

The term “depression” as used herein refers to a disturbance of mood and is characterized by a loss of interest or pleasure in normal everyday activities. People who are depressed may feel “down in the dumps” for weeks, months, or even years at a time. Some of the following symptoms may be symptoms of depression: persistent sad, anxious, or “empty” mood; feelings of hopelessness, pessimism; feelings of guilt, worthlessness, helplessness; loss of interest or pleasure in hobbies and activities that were once enjoyed, including sex; decreased energy, fatigue, being “slowed down”; difficulty concentrating, remembering, making decisions; insomnia, early-morning awakening, or oversleeping; appetite and/or weight loss or overeating and weight gain; thoughts of death or suicide; suicide attempts; restlessness, irritability; persistent physical symptoms that do not respond to treatment, such as headaches, digestive disorders, and chronic pain.

The term “epilepsy” as used herein refers a brain disorder in which clusters of nerve cells, or neurons, in the brain sometimes signal abnormally. In epilepsy, the normal pattern of neuronal activity becomes disturbed, causing strange sensations, emotions, and behavior or sometimes convulsions, muscle spasms, and loss of consciousness. Epilepsy is a disorder with many possible causes. Anything that disturbs the normal pattern of neuron activity—from illness to brain damage to abnormal brain development—can lead to seizures. Epilepsy may develop because of an abnormality in brain wiring, an imbalance of nerve signaling chemicals called neurotransmitters, or some combination of these factors. Having a seizure does not necessarily mean that a person has epilepsy. Only when a person has had two or more seizures is he or she considered to have epilepsy.

The term “seizure” as used herein refers to a transient alteration of behaviour due to the disordered, synchronous, and rhythmic firing of populations of brain neurones.

The term “migraine” as used herein means a disorder characterised by recurrent attacks of headache that vary widely in intensity, frequency, and duration. The pain of a migraine headache is often described as an intense pulsing or throbbing pain in one area of the head. It is often accompanied by extreme sensitivity to light and sound, nausea, and vomiting. Some individuals can predict the onset of a migraine because it is preceded by an “aura,” visual disturbances that appear as flashing lights, zig-zag lines or a temporary loss of vision. People with migraine tend to have recurring attacks triggered by a lack of food or sleep, exposure to light or hormonal irregularities (only in women). Anxiety, stress, or relaxation after stress can also be triggers. For many years, scientists believed that migraines were linked to the dilation and constriction of blood vessels in the head. Investigators now believe that migraine is caused by inherited abnormalities in genes that control the activities of certain cell populations in the brain. The International Headache Society (IHS, 1988) classifies migraine with aura (classical migraine) and migraine without aura (common migraine) as the major types of migraine.

The term “multiple sclerosis” (MS) as used herein is a chronic disease of the central nervous system in which gradual destruction of myelin occurs in patches throughout the brain or spinal cord or both, interfering with the nerve pathways. As more and more nerves are affected, a patient experiences a progressive interference with functions that are controlled by the nervous system such as vision, speech, walking, writing, and memory.

Activity in any of the above-mentioned indications can of course be determined by carrying out suitable clinical trials in a manner known to a person skilled in the relevant art for the particular indication and/or in the design of clinical trials in general.

For treating diseases, compounds of formula (I) or their pharmaceutically acceptable salts may be employed at an effective daily dosage and administered in the form of a pharmaceutical composition.

Therefore, another embodiment of the present invention concerns a pharmaceutical composition comprising an effective amount of a compound of formula (I) or a pharmaceutically acceptable salt thereof in combination with a pharmaceutically acceptable diluent or carrier.

To prepare a pharmaceutical composition according to the invention, one or more of the compounds of formula (I) or a pharmaceutically acceptable salt thereof is intimately admixed with a pharmaceutical diluent or carrier according to conventional pharmaceutical compounding techniques known to the skilled practitioner.

Suitable diluents and carriers may take a wide variety of forms depending on the desired route of administration, e.g., oral, rectal, parenteral or intranasal.

Pharmaceutical compositions comprising compounds according to the invention can, for example, be administered orally, parenterally, i.e., intravenously, intramuscularly or subcutaneously, intrathecally, by inhalation or intranasally.

Pharmaceutical compositions suitable for oral administration can be solids or liquids and can, for example, be in the form of tablets, pills, dragees, gelatin capsules, solutions, syrups, chewing-gums and the like.

To this end the active ingredient may be mixed with an inert diluent or a non-toxic pharmaceutically acceptable carrier such as starch or lactose. Optionally, these pharmaceutical compositions can also contain a binder such as microcrystalline cellulose, gum tragacanth or gelatine, a disintegrant such as alginic acid, a lubricant such as magnesium stearate, a glidant such as colloidal silicon dioxide, a sweetener such as sucrose or saccharin, or colouring agents or a flavouring agent such as peppermint or methyl salicylate.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the active ingredient, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, chelating agents such as ethylene diamine-tetra-acetic acid, buffers such as acetates, citrates or phosphates and agents for adjusting the osmolarity, such as sodium chloride or dextrose.

These pharmaceutical forms are prepared using methods which are routinely used by pharmacists.

The amount of active ingredient in the pharmaceutical compositions can fall within a wide range of concentrations and depends on a variety of factors such as the patient's sex, age, weight and medical condition, as well as on the method of administration. Thus the quantity of compound of formula (I) in compositions for oral administration is at least 0.5% by weight and can be up to 80% by weight with respect to the total weight of the composition.

For the preferred oral compositions, the daily dosage is in the range 3 to 3000 milligrams (mg) of compounds of formula (I).

In compositions for parenteral administration, the quantity of compound of formula (I) present is at least 0.5% by weight and can be up to 33% by weight with respect to the total weight of the composition. For the preferred parenteral compositions, the dosage unit is in the range 3 mg to 3000 mg of compounds of formula (I).

The daily dose can fall within a wide range of dosage units of compound of formula (I) and is generally in the range 3 to 3000 mg. However, it should be understood that the specific doses can be adapted to particular cases depending on the individual requirements, at the physician's discretion.

The following examples illustrate how the compounds covered by formula (I) may be synthesized. They are provided for illustrative purposes only and are not intended, nor should they be construed, as limiting the invention in any manner. Those skilled in the art will appreciate that routine variations and modifications of the following examples can be made without exceeding the spirit or scope of the invention.

NMR spectra are recorded on a BRUKER AVANCE 400 NMR Spectrometer fitted with a Linux workstation running XWIN NMR 3.5 software and a 5 mm inverse ¹H/BB probehead, or BRUKER DRX 400 NMR fitted with a SG Fuel running XWIN NMR 2.6 software and a 5 mm inverse geometry ¹H/¹³C/¹⁹F triple probehead. The compound is studied in d₆-dimethylsulfoxide (or d₃-chloroform) solution at a probe temperature of 313 K or 300 K and at a concentration of 10 mg/ml. The instrument is locked on the deuterium signal of d₆-dimethylsulfoxide (or d₃-chloroform). Chemical shifts are given in ppm downfield from TMS (tetramethylsilane) taken as internal standard.

HPLC analyses are performed using one of the following systems:
an Agilent 1100 series HPLC system mounted with an INERTSIL ODS 3 C18, DP 5 μm, 250×4.6 mm column. The gradient runs from 100% solvent A (acetonitrile, water, phosphoric acid (5/95/0.001, v/v/v)) to 100% solvent B (acetonitrile, water, phosphoric acid (95/5/0.001, v/v/v)) in 6 min with a hold at 100% B of 4 min. The flow rate is set at 2.5 ml/min. The chromatography is carried out at 35° C.
a HP 1090 series HPLC system mounted with a HPLC Waters Symetry C18, 250×4.6 mm column. The gradient runs from 100% solvent A (methanol, water, phosphoric acid (15/85/0.001M, v/v/M)) to 100% solvent B (methanol, water, phosphoric acid (85/15/0.001 M, v/v/M)) in 10 min with a hold at 100% B of 10 min. The flow rate is set at 1 ml/min. The chromatography is carried out at 40° C.

Mass spectrometric measurements in LC/MS mode are performed as follows:

HPLC Conditions

Analyses are performed using a WATERS Alliance HPLC system mounted with an INERTSIL ODS 3, DP 5 μm, 250×4.6 mm column.

The gradient runs from 100% solvent A (acetonitrile, water, trifluoroacetic acid (10/90/0.1, v/v/v)) to 100% solvent B (acetonitrile, water, trifluoroacetic acid (90/10/0.1, v/v/v)) in 7 min with a hold at 100% B of 4 min. The flow rate is set at 2.5 ml/min and a split of 1/25 is used just before API source.

MS Conditions

Samples are dissolved in acetonitrile/water, 70/30, v/v at the concentration of about 250 μg/ml. API spectra (+ or −) are performed using a FINNIGAN LCQ ion trap mass spectrometer. APCI source operated at 450° C. and the capillary heater at 160° C. ESI source operated at 3.5 kV and the capillary heater at 210° C.

Mass spectrometric measurements in DIP/EI mode are performed as follows: samples are vaporized by heating the probe from 50° C. to 250° C. in 5 min. EI (Electron Impact) spectra are recorded using a FINNIGAN TSQ 700 tandem quadrupole mass spectrometer. The source temperature is set at 150° C.

Mass spectrometric measurements on a TSQ 700 tandem quadrupole mass spectrometer (Finnigan MAT) in GC/MS mode are performed with a gas chromatograph model 3400 (Varian) fitted with a split/splitless injector and a DB-5MS fused-silica column (15 m×0.25 mm I.D., 1 μm) from J&W Scientific. Helium (purity 99.999%) is used as carrier gas. The injector (CTC A200S autosampler) and the transfer line operate at 290 and 250° C., respectively. Sample (1 μl) is injected in splitless mode and the oven temperature is programmed as follows: 50° C. for 5 min., increasing to 280° C. (23° C./min) and holding for 10 min. The TSQ 700 spectrometer operates in electron impact (EI) or chemical ionization (Cl/CH₄) mode (mass range 33-800, scan time 1.00 sec). The source temperature is set at 150° C.

High resolution mass spectrometry measurements are run on a Waters LCT Time of flight mass spectrometer equipped with an ESI source and a Waters Acquity UPLC (column: BEH C18 (1.7 μm, 2.1×50 mm)) with diode array detector. The gradient runs from 98% solvent A (aqueous ammonium formate (63 mg/l), 30% aqueous ammonia (50 μl/l)) to 95% acetonitrile and back in 6 min. The source parameters are as follows: ESI capillary voltage 2.5 kV, cone voltage 135 V, source block temperature 135° C., desolvation temperature 350° C., cone gas flow 20 L/Hr (Nitrogen), desolvation Gas flow 800 L/Hr. The detector is set with a flight tube at 7.2 KV and an MCP detector at 2,500 V.

Specific rotation is recorded on a Perkin-Elmer 341 polarimeter. The angle of rotation is recorded at 25° C. on 1% solutions in methanol, at 589 nm.

Melting points are determined on a Büchi 535 or 545 Tottoli-type fusionometer, and are not corrected, or by the onset temperature on a Perkin Elmer DSC 7.

Preparative chromatographic separations are performed on silicagel 60 Merck, particle size 15-40 μm, reference 1.15111.9025, using Novasep axial compression columns (80 mm i.d.), flow rates between 70 and 150 ml/min. Amount of silicagel and solvent mixtures as described in individual procedures. Reverse phase separations are carried out using 500 g of either Kromasil C18 10 μm silicagel (acidic or neutral conditions) or Phenomenex Gemini C18 10 μM (basic conditions) in 8-cm ID columns with a flow rate of 150 ml/min. Products are detected at 215 nm unless otherwise specified.

Preparative Chiral Chromatographic separations are performed on a DAICEL Chiralpak AD 20 μm, 100*500 mm column using an in-house build instrument with various mixtures of lower alcohols and C5 to C8 linear, branched or cyclic alkanes at ±350 ml/min. Solvent mixtures as described in individual procedures.

Experiments requiring microwave irradiation are performed on a Biotage Initiator Sixty microwave oven upgraded with version 2.0 of the operating software. Experiments are run to reach the required temperature as quickly as possible (maximum irradiation power: 400 W, no external cooling).

### EXAMPLE 1

### Synthesis of 1-{trans-3-[4-(piperidin-1-ylcarbonyl)phenoxy]cyclobutyl}piperidine 3

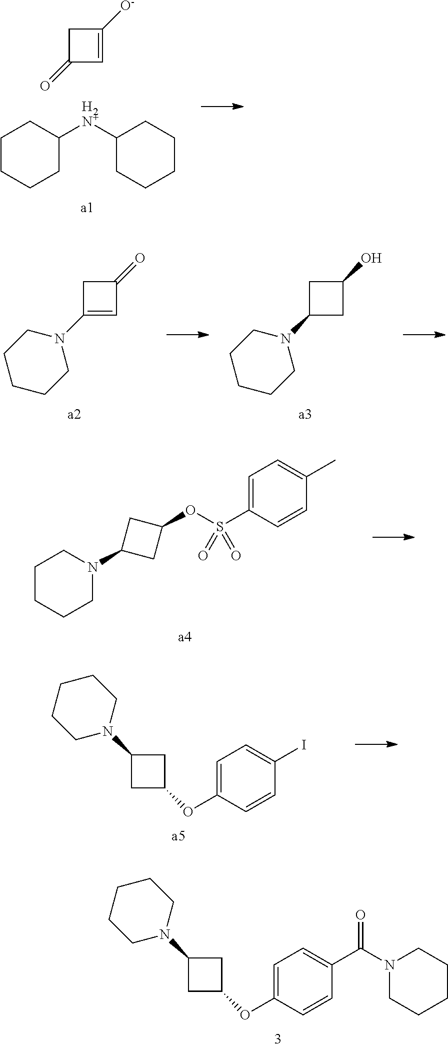

1.1 Synthesis of 3-piperidin-1-ylcyclobut-2-en-1-one a2.

Trifluoroacetic acid (64 ml, 0.825 mol, 1.1 eq) is added over 10 minutes to a stirred suspension of N-cyclohexylcyclohexanaminium 3-oxocyclobut-1-en-1-olate a1 (200 g, 0.75 mol, 1 eq) in dioxane (1 l). After 4 hours stirring at room temperature, the resulting suspension is filtered and washed with dioxane (300 ml). The filtrate is then stirred at room temperature and treated dropwise with piperidine (96 ml, 0.975 mol, 1.3 eq) while maintaining the temperature below 30° C. throughout the addition (20 minutes) with a water bath. The dioxane is then removed under reduced pressure and the resulting oil is taken up in dichloromethane (400 ml). The organic layer is washed with a 1N aqueous hydrochloric acid solution (400 ml), water (400 ml), an aqueous saturated solution of sodium hydrogencarbonate (400 ml) and brine (400 ml). The organic layer is dried over magnesium sulfate and concentrated to yield 90.7 g of a red solid. The solid is then purified by chromatography over silicagel (dichloromethane/methanol/ammonia 98:1.8:0.2) to afford 74.8 g of 3-piperidin-1-ylcyclobut-2-en-1-one a2.

Yield: 66%.

¹H NMR (CDCl₃) δ: 4.47 (s, 1 H), 3.22 (m, 4 H), 2.95 (s, 2 H), 1.53 (m, 6 H).

1.2 Synthesis of cis-3-piperidin-1-ylcyclobutanol a3.

A solution of 3-piperidin-1-ylcyclobut-2-en-1-one a2 (10 g, 66.1 mmol, 1 eq) in ethanol (200 ml) is treated with portions of sodium borohydride (8.76 g, 231 mmol, 3.5 eq). At the end of the addition, the mixture is stirred at 50° C. for 12 h, cooled down to 20° C. and treated with acetone (20 ml). The solvents are removed under reduced pressure to afford a yellow oil which is diluted with ethyl acetate (200 ml). This organic layer is washed with an aqueous saturated solution of sodium hydrogencarbonate (100 ml), water (100 ml) and brine (100 ml), then concentrated under reduced pressure. The residual oil is purified by chromatography over silicagel, (dichloromethane/methanol/ammonia 95:4.5:0.5) to afford 8 g of cis-3-piperidin-1-ylcyclobutanol a3 as a white solid.

Yield: 78%.

¹H NMR (CDCl₃) δ: 3.81 (m, 3 H), 2.38 (m, 2 H), 2.06 (m, 4 H), 1.69 (m, 2 H), 1.43 (m, 4 H), 1.29 (bs, 2 H).

1.3 Synthesis of cis-3-piperidin-1-ylcyclobutyl 4-methylbenzenesulfonate a4.

A solution of cis-3-piperidin-1-ylcyclobutanol a3 (1.0 g, 6.44 mmol, 1.0 eq) and N-methylimidazole (1.03 ml, 12.88 mmol, 2.0 eq) in dichloromethane (10 ml) is treated with p-toluenesulfonyl chloride (2.1 g, 10.95 mmol, 1.7 eq). The mixture is stirred at 20° C. for 48 h, washed with an aqueous saturated solution of sodium hydrogencarbonate (10 ml), dried over magnesium sulfate and concentrated to afford 1.8 g of a red oil. This oil is purified by chromatography over silicagel (dichloromethane/methanol/ammonia 99:0.9:0.1) to yield 1.1 g of cis-3-piperidin-1-ylcyclobutyl 4-methylbenzenesulfonate a4 as an orange solid.

Yield: 55%.

LC-MS (MH⁺): 310.

1.4 Synthesis of 1-[trans-3-(4-iodophenoxy)cyclobutyl]piperidine a5.

A solution of para-iodophenol (15.46 g, 70.29 mmol, 1.5 eq) in dry N,N-dimethylacetamide (65 ml) is treated with sodium hydride (60% dispersion in mineral oil, 3.37 g, 84.35 mmol, 1.8 eq) under an argon atmosphere. After 30 minutes, cis-3-piperidin-1-ylcyclobutyl 4-methylbenzenesulfonate a4 (14.5 g, 46.86 mmol, 1 eq) is added and the mixture is stirred at 80° C. overnight. The mixture is concentrated under reduced pressure, diluted with ethyl acetate (200 ml) and washed twice with an aqueous saturated solution of sodium hydrogencarbonate. The organic layer is then dried over magnesium sulfate and concentrated under reduced pressure. The residue is purified by chromatography over silicagel (dichloromethane/ethanol 98:2 to 93:7) to afford 11.5 g of 1-[trans-3-(4-iodophenoxy)cyclobutyl]piperidine a5 as an orange oil.

Yield: 69%.

LC-MS (MH⁺): 358.

1.5 Synthesis of 1-{trans-3-[4-(piperidin-1-ylcarbonyl)phenoxy]cyclobutyl}piperidine 3.

1,8-Diazabicyclo[5.4.0]undec-7-ene (0.92 ml, 6.17 mmol, 3.15 eq) is added to a solution of 1-[trans-3-(4-iodophenoxy)cyclobutyl]piperidine a5 (0.7 g, 1.96 mmol, 1 eq), piperidine (0.58 ml, 5.88 mmol, 3 eq), palladium acetate (88 mg, 0.39 mmol, 0.2 eq), molybdenumhexacarbonyl (569 mg, 2.16 mmol, 1.1 eq) and 200 mg of molecular sieves at 0° C. The mixture is stirred under micro-wave irradiation at 125° C. during 20 minutes, filtered over celite and concentrated under reduced pressure. The residue is taken up in dichloromethane and washed three times with water, and once with a saturated aqueous solution of sodium chloride. The organic layer is dried over magnesium sulfate and concentrated under reduced pressure. The crude residue is taken up into ethyl acetate and filtered. The supernatant is concentrated and purified by chromatography over silicagel (dichloromethane/methanol/ammonia 97:2.7:0.3) to give 263 mg of 1-{trans-3-[4-(piperidin-1-ylcarbonyl)phenoxy]cyclobutyl}piperidine 3 as a pink solid.

Yield: 40%.

LC-MS (MH⁺): 343.

Compounds 1, 2, 4, 5, 6, 7, 8, 9, 10, 11, 12, 13 and 14 may be synthesized according to the same method.

### EXAMPLE 2

### Synthesis of 4-(4-{[trans-3-(2-methylpyrrolidin-1-yl)cyclobutyl]oxy}benzoyl)morpholine 17 and 4-{[4-({trans-3-[(2S)-2-methylpyrrolidin-1-yl]cyclobutyl}oxy)phenyl]carbonothioyl}morpholine 18

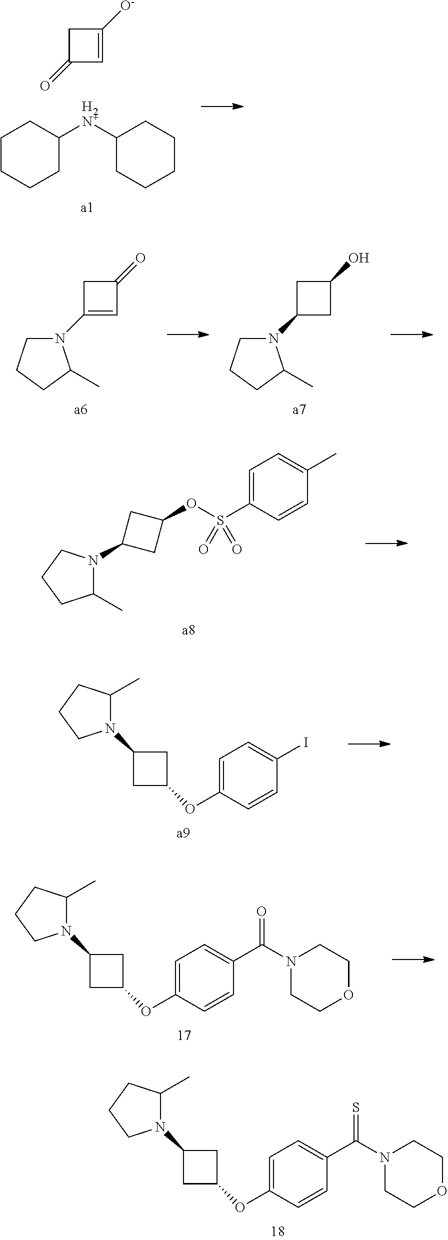

2.1 Synthesis of 3-(2-methylpyrrolidin-1-yl)cyclobut-2-en-1-one a6.

Trifluoroacetic acid (15.75 ml, 0.207 mol, 1.1 eq) is added over 10 minutes to a stirred suspension of N-cyclohexylcyclohexanaminium 3-oxocyclobut-1-en-1-olate (50 g, 0.19 mol, 1 eq) in dioxane (250 ml). After 20 hours stirring at room temperature, the resulting suspension is filtered and washed with dioxane (40 ml). The filtrate is stirred at room temperature and treated dropwise with 2-methylpyrrolidine (20 g, 0.245 mol, 1.3 eq) while maintaining the temperature below 30° C. throughout the addition (20 minutes) with a water bath. The mixture is then allowed to stir 24 h at 20° C. The dioxane is removed under reduced pressure and the resulting oil is purified by chromatography over silicagel (dichloromethane/methanol/ammonia 98:1.8:0.2) to afford 7.256 g of 3-(2-methylpyrrolidin-1-yl)cyclobut-2-en-1-one a6 as a yellow oil.

Yield: 25%.

RMN ¹H (CDCl₃): δ 4.55 (d, J=6.8 Hz, 1 H), 3.94 (m, 1 H), 3.81 (m, 1 H), 3.53 (m, 2 H), 3.35 (m), 3.32 (m, 1 H), 3.18 (s, 1 H), 1.74 (m, 1 H), 1.26 (dd, J=9.2, 6.6 Hz, 3 H).

2.2 Synthesis of cis-3-(2-methylpyrrolidin-1-yl)cyclobutanol a7.

A solution of aqueous sodium hydroxide (46%, 1.5 ml, 0.5 eq) in methanol (85 ml) is treated with sodium borohydride (6.2 g, 0.16 mol, 3.4 eq), then a solution of 3-(2-methylpyrrolidin-1-yl)cyclobut-2-en-1-one a6 (7.26 g, 78 mmol, 1 eq) in methanol (40 ml) is added dropwise over 20 minutes. The mixture is stirred at 20° C. for 1h30, then at 56° C. overnight and concentrated under reduced pressure. The residual paste is dissolved in water (40 ml) and extracted with dichloromethane (2×40 ml, then 20 ml). The organic layers are pooled, washed with a 10% aqueous solution of sodium hydroxide (30 ml) and dried over magnesium sulphate. After concentration, the residual oil (6.35 g) still contains the starting material. It is then resubmitted to the above-mentioned reacting conditions and treated in the same way to afford crude cis-3-(2-methylpyrrolidin-1-yl)cyclobutanol a7 as an oil (5.35 g). This compound is used in the next step without any further purification.

2.3 Synthesis of cis-3-(2-methylpyrrolidin-1-yl)cyclobutyl 4-methylbenzenesulfonate a8.

A solution of cis-3-(2-methylpyrrolidin-1-yl)cyclobutanol a7 (2.67 g, 17.2 mmol, 1.0 eq) and N-methylimidazole (1.51 ml, 19 mmol, 1.1 eq) in ethyl acetate (45 ml) is treated with p-toluenesulfonyl chloride (3.94 g, 20.7 mmol, 1.2 eq). The mixture is stirred at 20° C. for 2 h and treated with a saturated aqueous solution of ammonium chloride (45 ml). The aqueous phase is extracted with dichloromethane (90 ml), the organic phase is dried over magnesium sulfate and concentrated under vaduum to afford 3.45 g of a red oil. This oil is purified (twice) by chromatography over silicagel (gradient: dichloromethane/methanol 98:2 to 90:10) to afford 2.27 g of cis-3-(2-methylpyrrolidin-1-yl)cyclobutyl 4-methylbenzenesulfonate a8 as an orange solid.

Yield: 42%.

LC-MS (MH⁺): 310.

2.4 Synthesis of 1-[trans-3-(4-iodophenoxy)cyclobutyl]-2-methylpyrrolidine a9.

A solution of para-iodophenol (2.035 g, 9.2 mmol, 1.5 eq) in dry N,N-dimethylacetamide (100 ml) is treated with sodium hydride (60% dispersion in mineral oil, 444 mg, 11 mmol, 1.8 eq) under an argon atmosphere. After 30 minutes, cis-3-(2-methylpyrrolidin-1-yl)cyclobutyl 4-methylbenzenesulfonate a8 (1.908 g, 6.2 mmol, 1 eq) is added and the mixture is stirred at 60° C. overnight. The mixture is diluted with ethyl acetate (800 ml) and washed twice with a saturated aqueous solution of sodium chloride. The organic layer is dried over magnesium sulfate and concentrated under reduced pressure. The residue is purified by chromatography over silicagel (gradient: dichloromethane/methanol/ammonia 98/1.8/0.2 to 90/9/1) to afford 890 mg of 1-[trans-3-(4-iodophenoxy)cyclobutyl]-2-methylpyrrolidine a9 as an orange oil.

Yield: 69%.

LC-MS (MH⁺): 358.

(2S)-1-[trans-3-(4-iodophenoxy)cyclobutyl]-2-methylpyrrolidine a10 and (2R)-1-[trans-3-(4-iodophenoxy)cyclobutyl]-2-methylpyrrolidine all are obtained by chiral chromatography of the racemic mixture a9 (chiralcel OJ-H; iso-propanol/benzene/diethylamine 10/90/1).

2.5 Synthesis of 4-(4-{[trans-3-(2-methylpyrrolidin-1-yl)cyclobutyl]oxy}benzoyl)morpholine 17.

1,8-Diazabicyclo[5.4.0]undec-7-ene (666 mg, 4.4 mmol, 2.5 eq) is added to a solution of 1-[trans-3-(4-iodophenoxy)cyclobutyl]-2-methylpyrrolidine a9 (625 mg, 1.7 mmol, 1 eq), morpholine (381 mg, 4.4 mmol, 2.5 eq), palladium acetate (78 mg, 0.3 mmol, 0.2 eq), molybdenumhexacarbonyl (508 mg, 1.9 mmol, 1.1 eq) and molecular sieves (220 mg) at 0° C. The mixture is stirred under micro-wave irradiation at 105° C. during 20 minutes, filtered over celite and concentrated under reduced pressure. The residue is taken up in ethyl acetate (100 ml) and washed with water (50 ml). The aqueous phase is back-extracted with ethyl acetate. The pooled organic layers are dried over magnesium sulfate and concentrated under reduced pressure. The residue is purified by chromatography over silicagel (dichloromethane/methanol/ammonia 90:9:1) to afford 100 mg of a yellow oil. This oil is purified further by reverse phase HPLC (gradient: water/acetonitrile/8% ammonium carbonate from 85:5:10 to 5:95:0) to give 61.4 mg of 4-(4-{[trans-3-(2-methylpyrrolidin-1-yl)cyclobutyl]oxy}benzoyl) morpholine 17 as a yellow oil.

Yield: 10%.

LC-MS (MH⁺): 343.

4-[4-({trans-3-[(2R)-2-methylpyrrolidin-1-yl]cyclobutyl}oxy)benzoyl]morpholine 15 and 4-[4-({trans-3-[(2S)-2-methylpyrrolidin-1-yl]cyclobutyl}oxy)benzoyl]morpholine 16 may be synthesized according to the same method.

2.6 Synthesis of 4-{[4-({trans-3-[(25)-2-methylpyrrolidin-1-yl]cyclobutyl}oxy)phenyl]carbonothioyl}morpholine 18.

A solution of 4-(4-{[trans-3-(2-methylpyrrolidin-1-yl)cyclobutyl]oxy}benzoyl)morpholine 17 in tetrahydrofuran (2 ml) is treated with Lawesson's reagent (1.5 eq, 0.3 mmol, 116.2 mg). The reaction mixture is stirred at 20° C. for 12 h, filtered and the resulting solution is filtered over Statosphere PL-SO3H acidic resin (0.6 mmol). The resin is rinsed with tetrahydrofuran and the combined organic phases are concentrated under reduced pressure. The resulting solid is purified by reverse phase HPLC (gradient: water/acetonitrile/8% ammonium carbonate 85:5:10 to 5:95:0) to afford 16 mg of a yellow oil.

Yield: 54%.

LC-MS (MH⁺): 361.

### EXAMPLE 3

### Synthesis of 4-{4-[(trans-3-piperidin-1-ylcyclobutyl)thio]benzoyl}morpholine 43

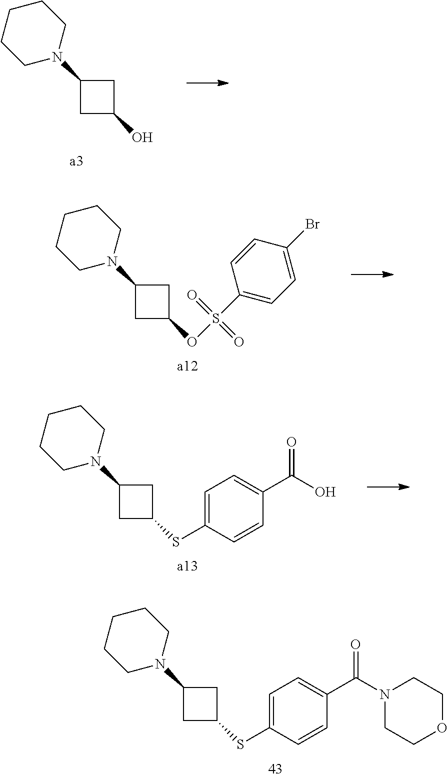

3.1 Synthesis of cis-3-(piperidin-1-yl)cyclobutyl 4-bromobenzenesulfonate a12.

A solution of cis-3-piperidin-1-ylcyclobutanol a3 (310 mg, 2 mmol, 1 eq.) in ethyl acetate (10 ml) is treated with 4-bromobenzenesulfonyl chloride (613 mg, 2.4 mmol, 1.2 eq) and N-methylimidazole (240 μl, 3 mmol, 1.5 eq). The mixture is stirred for 12 h at room temperature. The reaction mixture is filtered and the precipitate is rinsed with ethyl acetate. The solid is dissolved in ethyl acetate and washed with saturated sodium hydrogencarbonate and saturated ammonium chloride. The organic phase is dried over magnesium sulphate to yield 543 mg of cis-3-(piperidin-1-yl)cyclobutyl 4-bromobenzenesulfonate a12 as a yellow oil.

Yield: 72%.

LC-MS (MH⁺): 374/376.

3.2 Synthesis of 4-{[trans-3-(piperidin-1-yl)cyclobutyl]sulfanyl}benzoic acid a13.

A solution of 4-mercaptobenzoic acid (0.73 g, 4.71 mmol, 2.25 eq) in dry N,N-dimethylformamide (30 ml) is treated with sodium thiosulfate (0.7 g, 4.03 mmol, 1.93 eq) over molecular sieves (4A beads, 0.53 g) for 1 hour. Sodium hydride (60% dispersion in oil, 0.24 g, 6.02 mmol, 2.87 eq) is added, then cis-3-(piperidin-1-yl)cyclobutyl 4-bromobenzenesulfonate a12 (0.78 g, 2.09 mmol, 1 eq) and the mixture is stirred 2 h at 20° C. and 12 h at 50° C. The mixture is concentrated to dryness. The resulting solid is triturated in ethyl acetate (25 ml), acetonitrile (2×25 ml) and dichloromethane (25 ml). The solid is then taken up in methanol (3×20 ml) and filtered. The methanol phases are pooled and concentrated to dryness. The resulting solid is then purified by chromatography over silicagel (gradient: dichloromethane/methanol 100:0 to 0:100) to afford 0.56 g of 4-{[trans-3-(piperidin-1-yl)cyclobutyl]sulfanyl}benzoic acid a13.

Yield: 99%.

LC-MS (MH⁺): 292.

3.3 Synthesis of 4-{4-[(trans-3-piperidin-1-ylcyclobutyl)thio]benzoyl} morpholine 43.

A solution of O-benzotriazol-1-yl-N,N,N′,N′-tetramethyluronium tetrafluoroborate (1.36 g, 4.25 mmol, 2.6 eq)) in N,N-dimethylformamide (20 ml) is treated with 4-{[trans-3-(piperidin-1-yl)cyclobutyl]sulfanyl}benzoic acid a13 (0.45 g, 1.6 mmol, 1 eq) and stirred 10 minutes at 20° C. A mixture of diisopropylethylamine (0.5 ml, 3.02 mmol, 1.89 eq) and morpholine (0.3 ml, 3.44 mmol, 2.15 eq) is then added. The resulting mixture is stirred at 20° C. for 48 h, concentrated to 5 ml and treated with 1N hydrochloric acid (5 ml). The mixture is filtered and the solid washed with ethyl acetate (2×10 ml). The liquid phases are brought to pH 10 with potassium carbonate and the aqueous phase is extracted with ethyl acetate (3×25 ml). The combined organic layers are washed with water (2×25 ml), with brine (30 ml) and dried over magnesium sulphate. The organic phase is concentrated to dryness and the residual solid is purified by chromatography over silicagel (gradient: dichloromethane/methanol 100:0:0 to 90:9:1) to afford 4-{4-[(trans-3-piperidin-1-ylcyclobutyl)thio]benzoyl} morpholine 43.

Yield: 17%.

LC-MS (MH⁺): 361.

1-{4-[(trans-3-piperidin-1-ylcyclobutyl)thio]benzoyl}piperidin-4-one 44 may be synthesized according to the same method.

### EXAMPLE 4

### Synthesis of 1-{4-[(trans-3-piperidin-1-ylcyclobutyl)oxy]benzoyl}piperidine-4-carboxamide 28.

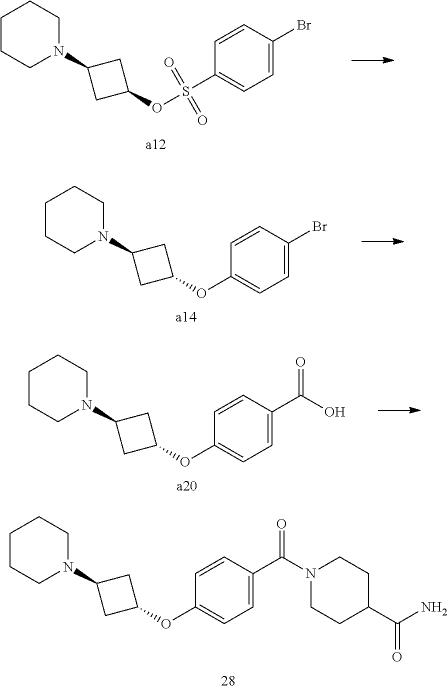

4.1 Synthesis of 1-[trans-3-(4-bromophenoxy)cyclobutyl]piperidine a14.

To a solution of para-bromophenol (2.5 mmol) in dry THF (30 ml) is added 15-Crown-5 (990 μl, 5 mmol, 2 eq) followed by 4 Å molecular sieves (2.1 g). The mixture is stirred at 30° C. for 30 minutes under an argon atmosphere. Sodium hydride 60% dispersion in mineral oil (210 mg, 5.25 mmol, 2.1 eq) is added slowly during a period of 1 hour. The mixture is then heated at 60° C. for an additional hour and cis-3-(piperidin-1-yl)cyclobutyl 4-bromobenzenesulfonate a12 (1029 mg, 2.75 mmol, 1.1 eq) is added. The mixture is stirred at reflux during 6 days. The mixture is cooled, filtered on celite and concentrated under vacuum. Ethyl acetate (250 ml) is added and the organic layer is washed twice with 2M NaOH (50 ml), brine (50 ml), dried over magnesium sulfate and concentrated under reduced pressure. The residue is purified by chromatography over silicagel (gradient: dichloromethane/methanol 100:0 to 95:5) to afford 1-[trans-3-(4-bromophenoxy)cyclobutyl]piperidine a14.

Yield: 81%.

LC-MS (MH⁺): 310-312.

The following compounds may be prepared according to the same method:

| | IUPAC Name | LC-MS (MH⁺) |
|---|---|---|
| a15 | 1-[trans-3-(4-bromo-2- | 328/330 |
| | fluorophenoxy)cyclobutyl]piperidine | |
| a16 | 1-[trans-3-(4-bromo-2- | 340/342 |
| | methoxyphenoxy)cyclobutyl]piperidine | |
| a17 | 1-[trans-3-(4-bromo-2- | 344/346/348 |
| | chlorophenoxy)cyclobutyl]piperidine | |
| a18 | 1-[trans-3-(4-bromo-3- | 328/330 |
| | fluorophenoxy)cyclobutyl]piperidine | |
| a19 | 1-[trans-3-(4-bromo-3- | 344/346/348 |
| | chlorophenoxy)cyclobutyl]piperidine | |

4.2 Synthesis of 4-{[trans-3-(piperidin-1-yl)cyclobutyl]oxy}benzoic acid a20.

A solution of 1-[trans-3-(4-bromophenoxy)cyclobutyl]piperidine a14 (1.4 mmol, 1 eq) in hexane (10 ml) at −70° C. is treated with butyllithium (2.5 M in hexane, 1.68 ml, 4.2 mmol, 3 eq) under an argon atmosphere. After 45 minutes of reaction, a large excess of solid carbon dioxide is added and the reaction temperature is allowed to reach slowly room temperature. The mixture is stirred 1 hour at room temperature, then water (2 ml) is added and the mixture is concentrated under reduced pressure. The residue is purified by basic reverse phase chromatography (gradient: acetonitrile/water/ammonium carbonate 5/95/0.1 to 95/5/0.1, 12 min) to afford 4-{[trans-3-(piperidin-1-yl)cyclobutyl]oxy}benzoic acid a20.

Yield: 66%.

LC-MS (MH⁺): 276.

3-fluoro-4-{[trans-3-(piperidin-1-yl)cyclobutyl]oxy}benzoic acid a21 may be synthesized according to the same method.

Yield: 32%.

LC-MS (MH⁺): 294.

Alternative method: synthesis of 3-methoxy-4-{[trans-3-(piperidin-1-yl)cyclobutyl]oxy}benzoic acid a22.

A solution of 1-[trans-3-(4-bromo-2-methoxyphenoxy)cyclobutyl]piperidine a16 (0.5 mmol) in dry tetrahydrofuran (10 ml) at −70° C. is treated with butyllithium (2.5M in hexane, 240 μl, 0.6 mmol, 1.2 eq) under an argon atmosphere. After 1 hour of reaction, ethyl chloroformate (57.4 μl, 0.6 mmol, 1.2 eq) is added and after 15 minutes at −70° C. the reaction temperature is allowed to reach slowly room temperature. Then saturated potassium carbonate (10 ml) and ethyl acetate (30 ml) are added and the product is extracted. The organic layer is washed with brine (10 ml), dried over magnesium sulfate and concentrated under reduced pressure. The residue is diluted in a mixture of methanol and water (1.5 ml:0.5 ml) and sodium hydroxide (2M, 300 μl, 0.6 mmol, 1.2 eq) is added. The mixture is stirred one night at room temperature. The residue is concentrated under reduced pressure. The residue is purified by chromatography over silica gel (gradient: dichloromethane/methanol 100:0 to 60/40) to afford 3-methoxy-4-{[trans-3-(piperidin-1-yl)cyclobutyl]oxy}benzoic acid a22.

Yield: 33%.

LC-MS (MH⁺): 306.

The following compounds may be prepared according to the same method:

| | IUPAC Name | LC-MS (MH⁺) |
|---|---|---|
| a23 | 3-chloro-4-{[trans-3-(piperidin-1- | 310/312 |
| | yl)cyclobutyl]oxy}benzoic acid | |
| a24 | 2-fluoro-4-{[trans-3-(piperidin-1- | 294 |
| | yl)cyclobutyl]oxy}benzoic acid | |
| a25 | 2-chloro-4-{[trans-3-(piperidin-1- | 310/312 |
| | yl)cyclobutyl]oxy}benzoic acid | |

4.3 Synthesis of 1-{4-[(trans-3-piperidin-1-ylcyclobutyl)oxy]benzoyl}piperidine-4-carboxamide 28.

A solution of 4-{[trans-3-(piperidin-1-yl)cyclobutyl]oxy}benzoic acid a20 (40 mg, 145 μmol) in DMF (500 μl) is treated with piperidine-4-carboxamide (20.5 mg, 160 μmol, 1.1 eq), O-benzotriazol-1-yl-N,N,N′,N′-tetramethyluronium tetrafluoroborate (49 mg, 152.5 μmol, 1.05 eq) and diisopropylethylamine (75.9 μl, 435 μM, 3 eq). After 1 day at room temperature, saturated sodium carbonate (2 ml) and ethyl acetate (2 ml) are added. The product is extracted and the aqueous phase extracted again with ethyl acetate (2 ml). The organic layers are washed with brine (1 ml) and concentrated under reduced pressure. The residue is purified by basic reverse phase chromatography (gradient: acetonitrile/water/ammonium hydrogenocarbonate 5/95/0.1 to 95/5/01, 12 min) to afford 33.9 mg of 1-{4-[(trans-3-piperidin-1-ylcylobutyl)oxy]benzoyl}piperidine-4-carboxamide 28.

Yield: 61%.

LC-MS (MH⁺): 385.

Compounds 19, 20, 21, 22, 23, 24, 25, 26, 27, 29, 30, 31, 32, 33, 34, 35, 36, 37, 38, 39, 40, 41 and 42 may be synthesized according to the same method.

Table I indicates the IUPAC name of the compound, the ion peak observed in mass spectrometry, the ¹H NMR description and melting point.

TABLE I

| n° | IUPAC name | MH⁺ | ¹H NMR δ (400 MHz, CDCl₃, ppm) | MP ° C. |
|---|---|---|---|---|
| 1 | 4-{4-[(trans-3-piperidin-1- | 345 | 7.36 (d, J = 8.0 Hz, 2H), 6.79 (d, J = 7.8 Hz, 2H), 4.77 (s, 1 | 135-136 |
| | ylcyclobutyl)oxy]benzoyl}morpholine | | H), 3.60 (m, 8 H), 2.99 (m, 1 H), 2.35 (m, 8 H), 1.53 (m, 6 H) | |
| 2 | 4,4-difluoro-1-{4-[(trans-3-piperidin-1- | 379 | 7.36 (d, J = 8.8 Hz, 2 H), 6.79 (d, J = 8.8 Hz, 2 H), 4.77 (m, | 145-146 |
| | ylcyclobutyl)oxy]benzoyl}piperidine | | 1 H), 3.72 (s, 4 H), 2.98 (m, 1 H), 2.41 (d, J = 6.0 Hz, 2 H), | |
| | | | 2.30 (m, 5 H), 2.00 (s, 4 H), 1.61 (m, 4 H), 1.46 (s, 2 H) | |
| 3 | 1-{trans-3-[4-(piperidin-1- | 343 | 7.34 (d, J = 8.8 Hz, 2 H), 6.77 (d, J = 8.8 Hz, 2 H), 4.76 (m, | 101.1-102.2 |
| | ylcarbonyl)phenoxy]cyclobutyl}piperidine | | 1 H), 3.54 (m, 3 H), 2.98 (t, J = 7.5 Hz, 1 H), 2.33 (m, 8 H), | |
| | | | 1.58 (m, 13 H) | |
| 4 | 1-isopropyl-4-{4-[(trans-3-piperidin-1- | 386 | 7.36 (d, J = 8.5 Hz, 2 H), 6.78 (d, J = 8.5 Hz, 2 H), 4.76 (m, | 98.5-99 |
| | ylcyclobutyl)oxy]benzoyl}piperazine | | 1 H), 2.99 (t, J = 7.3 Hz, 1 H), 2.72 (m, 1 H), 2.39 (m, 12 H), | |
| | | | 1.60 (m, 4 H), 1.46 (m, 2 H), 1.05 (d, J = 6.5 Hz, 6 H) | |
| 5 | 1-{4-[(trans-3-piperidin-1- | 359 | 7.35 (d, J = 8.8 Hz, 2 H), 6.78 (d, J = 8.5 Hz, 2 H), 4.77 (m, | 57.4-57.8 |
| | ylcyclobutyl)oxy]benzoyl}piperidin-4-ol | | 1 H), 3.96 (m, 2 H), 3.28 (s, 2 H), 3.01 (t, J = 7.3 Hz, 1 H), | |
| | | | 2.36 (m, 8 H), 1.85 (m, 4 H), 1.55 (m, 8 H) | |
| 6 | 4-{4-[(trans-3-piperidin-1- | 361 | 7.33 (d, J = 8.8 Hz, 2 H), 6.79 (d, J = 8.8 Hz, 2 H), 4.76 (m, | |
| | ylcyclobutyl)oxy]benzoyl}thiomorpholine | | 1 H), 3.86 (m, 4 H), 2.99 (t, J = 7.3 Hz, 1 H), 2.65 (s, 4 H), | |
| | | | 2.35 (m, 8 H), 1.60 (m, 4 H), 1.46 (d, J = 4.5 Hz, 2 H) | |
| 7 | 1-{trans-3-[4-(pyrrolidin-1- | 329 | 7.49 (d, J = 8.5 Hz, 2 H), 6.77 (d, J = 8.5 Hz, 2 H), 4.77 (m, | |
| | ylcarbonyl)phenoxy]cyclobutyl}piperidine | | 1 H), 3.63 (t, J = 6.8 Hz, 2 H), 3.48 (t, J = 6.3 Hz, 2 H), 2.98 | |
| | | | (m, 1 H), 2.34 (m, 7 H), 1.91 (m, 5 H), 1.60 (m, 4 H), 1.46 (d, | |
| | | | J = 4.5 Hz, 2 H) | |
| 8 | 4-{4-[(trans-3-piperidin-1- | 393 | 7.49 (s, 1 H), 7.43 (d, J = 8.5 Hz, 2 H), 6.86 (d, J = 8.5 Hz, 2 | 186.5-186.9 |
| | ylcyclobutyl)oxy]benzoyl}thiomorpholine | | H), 4.76 (m, 1 H), 3.86 (s, 4 H), 3.23 (s, 4 H), 2.86 (m, 1 H), | |
| | 1,1-dioxide | | 2.34 (m, 2 H), 2.16 (m, 6 H), 1.50 (m, 4 H), 1.39 (d, J = 4.5 | |
| | | | Hz, 2 H) | |
| 9 | 1-(trans-3-{4-[(3,3-difluoropyrrolidin-1- | 365 | (DMSO) 7.51 (m, 3 H), 6.86 (d, J = 8.8 Hz, 2 H), 4.77 (m, 1 | 115.2-115.8 |
| | yl)carbonyl]phenoxy}cyclobutyl)piperidine | | H), 3.89 (t, J = 12.8 Hz, 2 H), 3.70 (t, J = 7.3 Hz, 2 H), 2.86 | |
| | | | (m, 1 H), 2.39 (m, 4 H), 2.16 (m, 6 H), 1.50 (m, 4 H), 1.39 (d, | |
| | | | J = 4.8 Hz, 2 H) | |
| 10 | 1-acetyl-4-{4-[(trans-3-piperidin-1- | 386 | (DMSO) 7.49 (s, 1 H), 7.36 (d, J = 8.5 Hz, 2 H), 6.86 (d, J = | 159.2-159.8 |
| | ylcyclobutyl)oxy]benzoyl}piperazine | | 8.5 Hz, 2 H), 4.76 (m, 1 H), 3.47 (s, 7 H), 3.33 (s), 2.86 (t, J = | |
| | | | 6.3 Hz, 1 H), 2.35 (m, 2 H), 2.16 (m, 6 H), 2.02 (s, 3 H), | |
| | | | 1.49 (m, 4 H), 1.39 (d, J = 4.8 Hz, 2 H) | |
| 11 | 1-{4-[(trans-3-piperidin-1- | 331 | 7.57 (d, J = 8.5 Hz), 7.49 (s, 1 H), 6.84 (d, J = 8.8 Hz), 5.73 | 82.2-82.4 |
| | ylcyclobutyl)oxy]benzoyl}azetidin-3-ol | | (d, J = 6.0 Hz, 1 H), 4.76 (m, 1 H), 4.48 (m, 2 H), 4.21 (m, 1 | |
| | | | H), 4.02 (d, J = 1.3 Hz, 1 H), 3.76 (s, 1 H), 2.86 (t, J = 6.5 | |
| | | | Hz, 1 H), 2.35 (m, 2 H), 2.16 (m, 6 H), 1.50 (m, 4 H), 1.39 | |
| | | | (d, J = 4.8 Hz, 2 H) | |
| 12 | N-(4-chloropyridin-3-yl)-4-[(trans-3- | 386/388 | 9.71 (m, 1 H), 8.30 (m, 1 H), 8.18 (m, 1 H), 7.86 (m, 2 H), | 151.6 |
| | piperidin-1-ylcyclobutyl)oxy]benzamide | | 7.36 (m, 1 H), 6.90 (m, 2 H), 4.78 (m, 1 H), 2.99 (m, 1 H), | |
| | | | 2.18-2.52 (m, 8 H), 1.60 (d, J = 4.8 Hz, 4 H), 1.46 (s, 2 H) | |
| 13 | 4-[(trans-3-piperidin-1-ylcyclobutyl)oxy]- | 357 | (DMSO) 8.91 (t, J = 6.3 Hz, 1 H), 7.85 (d, J = 8.8 Hz, 2 H), | 152.9-153.4 |
| | N-(2,2,2-trifluoroethyl)benzamide | | 7.49 (s, 1 H), 6.89 (d, J = 8.8 Hz, 2 H), 4.79 (m, 1 H), 4.06 | |
| | | | (dd, J = 9.8, 6.3 Hz, 2 H), 2.86 (m, 1 H), 2.25 (m, 8 H), 1.45 | |
| | | | (m, 6 H) | |
| 14 | N-(3-aminopyridin-4-yl)-4-[(trans-3- | 367 | 9.54 (s, 1 H), 8.04 (s, 1 H), 7.95 (d, J = 8.8 Hz, 2 H), 7.91 (d, | |
| | piperidin-1-ylcyclobutyl)oxy]benzamide | | J = 5.5 Hz, 1 H), 6.91 (d, J = 8.8 Hz, 2 H), 6.65 (d, J = 5.5 | |
| | | | Hz, 1 H), 5.83 (s, 2 H), 4.82 (m, 1 H), 2.89 (m, 1 H), 2.39 (m, | |
| | | | 2 H), 2.24 (s, 3 H), 2.15 (m, 2 H), 1.51 (m, 4 H), 1.40 (d, J = | |
| | | | 4.5 Hz, 2 H) | |
| 15 | 4-[4-({trans-3-[(2R)-2-methylpyrrolidin-1- | 345 | 7.35 (d, J = 8.8 Hz, 2 H), 6.85 (d, J = 8.5 Hz, 2 H), 4.78 (m, | |
| | yl]cyclobutyl}oxy)benzoyl]morpholine | | 1 H), 3.58 (s, 4 H), 3.48 (s, 3 H), 2.88 (m, 1 H), 2.45 (m, 3 | |
| | | | H), 2.25 (m, 2 H), 2.11 (m, 1 H), 1.87 (m, 1 H), 1.64 (m, 2 | |
| | | | H), 1.32 (m, 1 H), 0.98 (d, J = 6.3 Hz, 3 H) | |
| 16 | 4-[4-({trans-3-[(2S)-2-methylpyrrolidin-1- | 345 | 7.36 (d, J = 8.6 Hz, 2 H), 6.79 (d, J = 8.6 Hz, 2 H), 4.77 (m, | |
| | yl]cyclobutyl}oxy)benzoyl]morpholine | | 1 H), 3.67 (m, 6 H), 3.40 (m, 1 H), 3.00 (m, 1 H), 2.47 (m, 4 | |
| | | | H), 2.27 (m, 2 H), 1.94 (m, 1 H), 1.79 (m, 1 H), 1.70 (m, 3 | |
| | | | H), 1.46 (m, 1 H), 1.09 (d, J = 6.1 Hz, 3 H) | |
| 17 | 4-(4-{[trans-3-(2-methylpyrrolidin-1- | 345 | 7.35 (d, J = 8.8 Hz, 2 H), 6.85 (d, J = 8.5 Hz, 2 H), 4.78 (m, | |
| | yl)cyclobutyl]oxy}benzoyl)morpholine | | 1 H), 3.58 (s, 4 H), 3.48 (s, 3 H), 2.88 (m, 1 H), 2.45 (m, 3 | |
| | | | H), 2.25 (m, 2 H), 2.11 (m, 1 H), 1.87 (m, 1 H), 1.64 (m, 2 | |
| | | | H), 1.32 (m, 1 H), 0.98 (d, J = 6.3 Hz, 3 H) | |
| 18 | 4-{[4-({trans-3-[(2S)-2-methylpyrrolidin-1- | 361 | 7.26 (m, 2 H), 6.74 (d, J = 8.5 Hz, 2 H), 4.78 (t, J = 6.3 Hz, 1 | |
| | yl]cyclobutyl}oxy)phenyl]carbonothioyl}mor- | | H), 4.42 (s, 2 H), 3.88 (s, 2 H), 3.66 (s, 4 H), 3.44 (quint, J = | |
| | pholine | | 7.5 Hz, 1 H), 3.06 (m, 1 H), 2.60 (m, 3 H), 2.36 (m, 3 H), | |
| | | | 1.97 (m, 1 H), 1.79 (m, 2 H), 1.51 (m, 1 H), 1.13 (d, J = 6.3 | |
| | | | Hz, 3 H) | |
| 19 | 4-{3-chloro-4-[(trans-3-piperidin-1- | 378.1710205* | 7.49 (d, J = 2.0 Hz, 1 H), 7.33 (dd, J = 8.3, 2.0 Hz, 1 H), | |
| | ylcyclobutyl)oxy]benzoyl}morpholine | | 6.94 (d, J = 8.5 Hz, 1 H), 4.85 (m, 1 H), 3.58 (s, 4 H), 3.47 | |
| | | | (d, J = 0.8 Hz, 3 H), 2.91 (m, 1 H), 2.36 (m, 2 H), 2.19 (m, 5 | |
| | | | H), 1.50 (d, J = 5.0 Hz, 4 H), 1.39 (m, 2 H) | |
| 20 | 1-{2-chloro-4-[(trans-3-piperidin-1- | 377.1506194* | | |
| | ylcyclobutyl)oxy]benzoyl}imidazolidin-4- | | | |
| | one | | | |
| 21 | 4-{2-fluoro-4-[(trans-3-piperidin-1- | 362.2005709* | | |
| | ylcyclobutyl)oxy]benzoyl}morpholine | | | |
| 22 | 1-[trans-3-(3-chloro-4-{[2- | 406.2023206* | 7.24 (d, J = 8.5 Hz, 1 H), 6.92 (d, J = 2.0 Hz, 1 H), 6.85 (dd, | |
| | (methoxymethyl)pyrrolidin-l - | | J = 8.3, 2.0 Hz, 1 H), 4.78 (m, 1 H), 4.19 (m, 1 H), 3.59 (dd, | |
| | yl]carbonyl}phenoxy)cyclobutyl]piperidine | | J = 9.0, 3.3 Hz, 1 H), 3.08 (m), 3.06 (m, 3 H), 3.01 (s), 2.87 | |
| | | | (m, 1 H), 2.35 (m, 3 H), 2.22 (d, J = 0.5 Hz, 3 H), 2.12 (m, 2 | |
| | | | H), 1.89 (m, 4 H), 1.73 (m, 1 H), 1.50 (m, 4 H), 1.39 (m, 2 H) | |
| | | | (signals of major rotamer, some signals obscured by | |
| | | | solvent) | |
| 23 | 2-{3-chloro-4-[(trans-3-piperidin-1- | 380.150285* | | |
| | ylcyclobutyl)oxy]benzoyl}isoxazolidin-4-ol | | | |
| 24 | 1-{trans-3-[3-chloro-4-(1,3-thiazolidin-3- | 380.1325265* | 7.32 (d, J = 8.3 Hz, 1 H), 6.96 (d, J = 2.3 Hz, 1 H), 6.87 (dd, | |
| | ylcarbonyl)phenoxy]cyclobutyl}piperidine | | J = 8.5, 2.3 Hz, 1 H), 4.79 (m, 1 H), 4.61 (s, 1 H), 4.22 (s, 1 | |
| | | | H), 3.81 (t, J = 6.5 Hz, 1 H), 3.44 (t, J = 6.0 Hz, 1 H), 3.08 (t, | |
| | | | J = 6.5 Hz, 1 H), 2.99 (t, J = 6.3 Hz, 1 H), 2.87 (m, 1 H), | |
| | | | 2.36 (m, 2 H), 2.22 (d, J = 1.3 Hz, 3 H), 2.13 (m, 2 H), 1.50 | |
| | | | (m, 4 H), 1.39 (m, 2 H) (some signals obscured by solvent) | |
| 25 | 1-{4-[(trans-3-piperidin-1- | 343.1895917* | | |
| | ylcyclobutyl)oxy]benzoyl}pyrazolidin-3- | | | |
| | one | | | |
| 26 | 2-{4-[(trans-3-piperidin-1- | 346.1892573* | | |
| | ylcyclobutyl)oxy]benzoyl}isoxazolidin-4-ol | | | |
| 27 | 4-{3-chloro-4-[(trans-3-piperidin-1- | 392.1866705* | 7.47 (d, J = 1.8 Hz, 1 H), 7.31 (dd, J = 8.3, 1.3 Hz, 1 H), | |
| | ylcyclobutyl)oxy]benzoyl}-1,4-oxazepane | | 6.93 (d, J = 8.3 Hz, 1 H), 4.84 (m, 1 H), 3.65 (m, 6 H), 3.46 | |
| | | | (d, J = 0.5 Hz, 2 H), 2.89 (m, 1 H), 2.36 (m, 2 H), 2.19 (m, 5 | |
| | | | H), 1.84 (m, 1 H), 1.75 (m, 1 H), 1.50 (m, 4 H), 1.40 (d, J = | |
| | | | 4.5 Hz, 2 H) (some signals obscured by solvent) | |
| 28 | 1-{4-[(trans-3-piperidin-1- | 385.2365419* | | |
| | ylcyclobutyl)oxy]benzoyl}piperidine-4- | | | |
| | carboxamide | | | |
| 29 | 1-{trans-3-[3-chloro-4-(pyrrolidin-1- | 362.1761058 | 7.27 (d, J = 8.5 Hz, 1 H), 6.92 (d, J = 2.3 Hz, 1 H), 6.84 (dd, | |
| | ylcarbonyl)phenoxy]cyclobutyl}piperidine | | J = 8.3, 2.3 Hz, 1 H), 4.77 (m, 1 H), 3.44 (t, J = 6.5 Hz, 2 H), | |
| | | | 3.09 (t, J = 6.5 Hz, 2 H), 2.87 (m, 1 H), 2.35 (m, 2 H), 2.23 | |
| | | | (m, 3 H), 2.12 (m, 2 H), 1.84 (m, 4 H), 1.50 (m, 4 H), 1.39 | |
| | | | (m, 2 H) (some signals obscured by solvent) | |
| 30 | 4-{2-fluoro-4-[(trans-3-piperidin-1- | 376.216221* | | |
| | ylcyclobutyl)oxy]benzoyl}-1,4-oxazepane | | | |
| 31 | 1-[trans-3-(4-{[2- | 372.2412929* | | |
| | (methoxymethyl)pyrrolidin-1- | | | |
| | yl]carbonyl}phenoxy)cyclobutyl]piperidine | | | |
| 32 | 4-{2-chloro-4-[(trans-3-piperidin-1- | 392.1866705* | 7.26 (dd, J = 8.5, 4.3 Hz, 1 H), 6.93 (dd, J = 5.0, 2.3 Hz, 1 | |
| | ylcyclobutyl)oxy]benzoyl}-1,4-oxazepane | | H), 6.85 (dt, J = 8.5, 2.8 Hz, 1 H), 4.78 (m, 1 H), 3.71 (m, 3 | |
| | | | H), 3.65 (t, J = 5.3 Hz, 2 H), 2.87 (m, 1 H), 2.35 (m, 2 H), | |
| | | | 2.22 (d, J = 1.3 Hz, 3 H), 2.13 (m, 2 H), 1.87 (m, 1 H), 1.68 | |
| | | | (m, 1 H), 1.50 (m, 4 H), 1.39 (d, J = 4.8 Hz, 2 H) (some | |
| | | | signals obscured by solvent) | |
| 33 | 2-{2-chloro-4-[(trans-3-piperidin-1- | 380.150285* | | |
| | ylcyclobutyl)oxy]benzoyl}isoxazolidin-4-ol | | | |
| 34 | 1-{3-fluoro-4-[(trans-3-piperidin-1- | 374.2005709* | 7.38 (dd, J = 11.8, 1.0 Hz, 1 H), 7.25 (m, 1 H), 6.99 (t, J = | |
| | ylcyclobutyl)oxy]benzoyl}piperidin-4-one | | 8.5 Hz, 1 H), 4.84 (m, 1 H), 3.73 (m, 4 H), 2.91 (m, 1 H), | |
| | | | 2.38 (m, 6 H), 2.20 (m, 6 H), 1.50 (m, 4 H), 1.39 (d, J = 4.5 | |
| | | | Hz, 2 H) | |
| 35 | 1-{trans-3-[4-(1,3-thiazolidin-3- | 346.1714988* | 7.49 (d, J = 8.8 Hz, 2 H), 6.86 (d, J = 8.5 Hz, 2 H), 4.77 (m, | |
| | ylcarbonyl)phenoxy]cyclobutyl}piperidine | | 1 H), 4.58 (s, 2 H), 3.77 (t, J = 6.3 Hz, 2 H), 3.01 (t, J = 6.3 | |
| | | | Hz, 2 H), 2.88 (m, 1 H), 2.36 (m, 2 H), 2.23 (m, 3 H), 2.14 | |
| | | | (m, 3 H), 1.50 (m, 4 H), 1.39 (m, 2 H) | |
| 36 | 1-{2-chloro-4-[(trans-3-piperidin-1- | 419.1975696* | | |
| | ylcyclobutyl)oxy]benzoyl}piperidine-4- | | | |
| | carboxamide | | | |
| 37 | 1-{4-[(trans-3-piperidin-1- | 343.1895917* | | |
| | ylcyclobutyl)oxy]benzoyl}imidazolidin-4- | | | |
| | one | | | |
| 38 | 1-[trans-3-(3-fluoro-4-{[2- | 390.2318711* | | |
| | (methoxymethyl)pyrrolidin-1- | | | |
| | yl]carbonyl}phenoxy)cyclobutyl]piperidine | | | |
| 39 | 1-{3-methoxy-4-[(trans-3-piperidin-1- | 373.2001564* | | |
| | ylcyclobutyl)oxy]benzoyl}imidazolidin-4- | | | |
| | one | | | |
| 40 | 4-{4-[(trans-3-piperidin-1- | 358.2256428* | 7.32 (d, J = 8.3 Hz, 2 H), 6.84 (d, J = 8.5 Hz, 2 H), 4.75 (m, | |
| | ylcyclobutyl)oxy]benzoyl}-1,4-oxazepane | | 1 H), 3.56 (m, 8 H), 2.86 (m, 1 H), 2.36 (m, 2 H), 2.23 (d, J = | |
| | | | 0.5 Hz, 3 H), 2.13 (m, 2 H), 1.74 (m, 2 H), 1.50 (m, 4 H), | |
| | | | 1.39 (d, J = 4.8 Hz, 2 H) | |
| 41 | 4-{2-chloro-4-[(trans-3-piperidin-1- | 378.1710205* | 7.27 (d, J = 8.5 Hz, 1 H), 6.94 (d, J = 2.3 Hz, 1 H), 6.86 (dd, | |
| | ylcyclobutyl)oxy]benzoyl}morpholine | | J = 8.3, 2.0 Hz, 1 H), 4.78 (m, 1 H), 3.62 (s, 4 H), 3.52 (t, J = | |
| | | | 4.3 Hz, 2 H), 3.13 (m, 2 H), 2.86 (m, 1 H), 2.35 (m, 2 H), | |
| | | | 2.22 (d, J = 1.3 Hz, 3 H), 2.12 (m, 2 H), 1.49 (m, 4 H), 1.39 | |
| | | | (d, J = 5.0 Hz, 2 H) (some signals obscured by solvent) | |
| 42 | 1-{trans-3-[3-fluoro-4-(1,3-thiazolidin-3- | 364.162077* | | |
| | ylcarbonyl)phenoxy]cyclobutyl}piperidine | | | |
| 43 | 4-{4-[(trans-3-piperidin-1- | 361 | 7.32 (m, 2 H), 7.18 (d, J = 8.3 Hz, 2 H), 3.85 (m, 1 H), 3.67 | |
| | ylcyclobutyl)thio]benzoyl}morpholine | | (m, 6 H), 3.05 (m, 1 H), 2.50 (m, 2 H), 2.24 (m, 3 H), 2.14 | |
| | | | (m, 2 H), 1.69 (m, 1 H), 1.58 (m, 4 H), 1.45 (m, 2 H) | |
| 44 | 1-{4-[(trans-3-piperidin-1- | 373 | 7.42 (d, J = 8.3 Hz, 2 H), 7.23 (d, J = 8.3 Hz, 2 H), 3.92 (s, 1 | |
| | ylcyclobutyl)thio]benzoyl}piperidin-4-one | | H), 3.74 (m, 4 H), 2.44 (s, 4 H), 2.21 (m, 3 H), 2.01 (m, 2 H), | |
| | | | 1.48 (d, J = 4.8 Hz, 4 H), 1.39 (m, 2 H) (some signals | |
| | | | obscured by solvent) | |

*High resolution mass spectrometry

### EXAMPLE 5

### Affinity for the Histamine H₃-Receptor; Inverse Agonism, Antagonism and Agonism Activity: [³⁵S]GTPγS-Binding Assay Human Histamine H₃-Receptor

Material and Methods

Reagents

Reagents and reference compounds are of analytical grade and may be obtained from various commercial sources. [³H]-N-α-methylhistamine (80-85 Ci/mmol) and [³⁵S]-GTPγS (1250 Ci/mmol) are purchased from Perkin Elmer (Belgium). Cell culture reagents are purchased from Cambrex (Belgium).

Test and reference compounds are dissolved in 100% DMSO to give a 1 mM stock solution. Final DMSO concentration in the assay does not exceed 1%.

A CHO cell line expressing the unspliced full length (445 AA) human H₃ histamine receptor may be obtained e.g. from Euroscreen S.A. (Belgium).

Cell Culture

Cells are grown in HAM-F12 culture media containing 10% fetal bovine serum, 100 IU/ml penicillin, 100 μg/ml streptomycin, 1% sodium pyruvate and 400 μg/ml of gentamycin. Cells are maintained at 37° C. in a humidified atmosphere composed of 95% air and 5% CO₂.

Membrane Preparation

Confluent cells are detached by 10 min incubation at 37° C. in PBS/EDTA 0.02%. The cell suspension is centrifuged at 1,500× g for 10 min at 4° C. The pellet is homogenized in a 15 mM Tris-HCl buffer (pH 7.5) containing 2 mM MgCl₂, 0.3 mM EDTA, 1 mM EGTA (buffer A). The crude homogenate is frozen in liquid nitrogen and thawed. DNAse (1 μl/ml) is then added and the homogenate is further incubated for 10 min at 25° C. before being centrifuged at 40,000× g for 25 min at 4° C. The pellet is resuspended in buffer A and washed once more under the same conditions. The final membrane pellet is resuspended, at a protein concentration of 1-3 mg/ml, in a 7.5 mM Tris-HCl buffer (pH 7.5) enriched with 12.5 mM MgCl₂, 0.3 mM EDTA, 1 mM EGTA and 250 mM sucrose and stored in liquid nitrogen until used.

Binding Assays

[³H]-N-α-methylhistamine Binding Assay

Affinity of compounds for human histamine H₃ receptors may be measured by competition with [³H]-N-α-methylhistamine. This binding assay may be performed on any H3 sequence, human or non-human. Briefly, membranes (20-40 μg proteins) expressing human H₃ histamine receptors are incubated at 25° C. in 0.5 ml of a 50 mM Tris-HCl buffer (pH 7.4) containing 2 mM MgCl₂, 0.2 nM [³H]-N-α-methyl-histamine and increasing concentrations of drugs. The non specific binding (NSB) is defined as the residual binding observed in the presence of 10 μM thioperamide or histamine. Membrane-bound and free radioligand are separated by rapid filtration through glass fiber filters presoaked in 0.1% PEI. Samples and filters are rinsed by at least 6 ml of ice-cold 50 mM Tris-HCl buffer (pH 7.4). The entire filtration procedure does not exceed 10 seconds per sample. Radioactivity trapped onto the filters is counted by liquid scintillation in a β-counter.

[³⁵S]-GTPγS Binding Assay

Stimulation (agonist) or inhibition (inverse agonist) of [³⁵S]-GTPγS binding to membrane expressing human H₃ histamine receptors is measured as described by Lorenzen et al. (Mol. Pharmacol. 1993, 44, 115-123) with a few modifications. Briefly, membranes (10-20 μg proteins) expressing human H₃ histamine receptors are incubated at 25° C. in 0.2 ml of a 50 mM Tris-HCl buffer (pH 7.4) containing 3 mM MgCl₂, 50 mM NaCl, 1 μM GDP, 2 μg saponin and increasing concentrations of drugs. After 15 min pre-incubation, 0.2 nM of [³⁵S]-GTPγS are added to the samples. The non specific binding (NSB) is defined as the residual binding observed in the presence of 100 μM Gpp(NH)p. Membrane-bound and free radioligand are separated by rapid filtration through glass fiber filters. Samples and filters are rinsed by at least 6 ml of ice-cold 50 mM Tris-HCl buffer (pH 7.4). The entire filtration procedure does not exceed 10 seconds per sample. Radioactivity trapped onto the filters is counted by liquid scintillation in a β-counter.

Data Analysis

Determination of pIC₅₀/pKi/pEC₅₀/pEC₅₀INV

Analysis

Raw data are analyzed by non-linear regression using XLfit™ (IDBS, United Kingdom) according to the following generic equation

B=MIN+[(MAX−MIN)/(1+(((10^{X})/10^{-pX50}))^{nH}))]

where:

B is the radioligand bound in the presence of the unlabelled compound (dpm),

MIN is the minimal binding observed (dpm)

MAX is maximal binding observed (dpm),

X is the concentration of unlabelled compound (log M),

pX₅₀ (−log M) is the concentration of unlabelled compound causing 50% of its maximal effect (inhibition or stimulation of radioligand binding). It stands for pIC₅₀ when determining the affinity of a compound for the receptor in binding studies with [³H]-N-α-methylhistamine, for pEC₅₀ for compounds stimulating the binding of [³⁵S]-GTPγS (agonists) and for pEC₅₀INV for compounds inhibiting the binding of [³⁵S]-GTPγS (inverse agonists).

n_{H} is the Hill coefficient.

pKi may be obtained by applying the following equation (Cheng and Prusoff, 1973, Biochem. Pharmacol., 22:3099-3108):

pKi=pIC₅₀+log (1+L/Kd)

where:

pKi is the unlabelled compound equilibrium dissociation constant (−log M),

L is the radioligand concentration (nM),

Kd is the radioligand equilibrium dissociation constant (nM).

Compounds of formula (I) according to the invention show pIC₅₀ values of at least 7, preferably greater than 8 for the histamine H₃ receptor.

Compounds of formula (I) according to the invention showed pEC₅₀INV values typically greater than 8 for the histamine H₃ receptor.

### EXAMPLE 6

### Antagonism Activity: Paced Isolated Guinea Pig Myenteric Plexus—Electric-Field Stimulation Assay

Material and Methods

Reagents

Stock solutions (10⁻² M) of compounds to be tested and further dilutions are freshly prepared in DMSO (WNR, Leuven, Belgium). All other reagents (R(−)-α-methylhistamine, mepyramine, ranitidine, propranolol, yohimbine and components of the Krebs' solution) are of analytical grade and obtained from conventional commercial sources.

Animals

Four week-old male Dunkin-Hartley guinea pigs (200-300 g) are supplied by Charles River (Sultfeld, Germany). All animals are ordered and used under protocol “orgisol-GP” approved by the UCB Pharma ethical committee. Animals are housed in the UCB animal facility in groups of 12, in stainless steel cages (75×50×30 cm) and allowed to acclimatise for a minimum of one week before inclusion in the study. Room temperature is maintained between 20 and 24° C. with 40 to 70% relative humidity. A light and dark cycle of 12 h is applied. Animals have free access to food and water.

Organ Preparation

The method is adapted from that described by Menkveld et al. in Eur. J. Pharmacol. 1990, 186, 343-347. Longitudinal myenteric plexus is prepared from the isolated guinea pig ileum. Tissues are mounted in 20-ml organ baths containing modified Krebs' solution with 10⁻⁷ M mepyramine, 10⁻⁵ M ranitidine, 10⁻⁵ M propranolol and 10⁻⁶ M yohimbine. The bathing solution is maintained at 37° C. and gassed with 95% O₂-5% CO₂. Tissues are allowed to equilibrate for a 60-min period under a resting tension of 0.5 g and an electrical field stimulation (pulses of 5-20 V, 1 ms and 0.1 Hz is applied during the whole experiment). Such a stimulation induces stable and reproductive twitch contractions. Isometric contractions are measured by force-displacement transducers coupled to an amplifier connected to a computer system (EMKA Technologies) capable of controlling (i) automatic data acquisition, (ii) bath washout by automatic fluid circulation through electrovalves at predetermined times or signal stability and (iii) automatic dilution/injection of drug in the bath at predetermined times or signal stability.

Protocol

After a 60 min-stabilisation period, tissues are stimulated twice with 10⁻⁶ M R(−)-α-methylhistamine at 30-min interval. After a 60-min incubation period in the presence of solvent or antagonist test compound, a cumulative concentration-response to R(−)-α-methylhistamine is elicited (10⁻¹⁰ à 10⁻⁴ M). Only one concentration of antagonist is tested on each tissue.

Data Analysis

An appropriate estimate of interactions between agonist and antagonist can be made by studying the family of curves observed in the absence or presence of increasing antagonist concentrations. The value of each relevant parameter of each concentration-response curve (pD₂ and Eₘₐₓ) is calculated by an iterative computer software (XLfit, IDBS, Guildford, UK) fitting the experimental data to the four parameter logistic equation. Antagonistic activity of the test substance is estimated by the calculation of pD′₂ and /or pA₂ values according to the methods described by Van Rossum et al. in Arch. Int. Pharmacodyn.Ther. 1963, 143, 299 and/or by Arunlakshana & Schild in Br. J. Pharmacol 1959, 14, 48

Results are expressed as the mean±SD. The number of observations is indicated as n.

Compounds of formula (I) according to the invention showed pA₂ values typically greater than or equal to 8 for the histamine H₃ receptor.

### EXAMPLE 7

### hERG Study

This is an in vitro electrophysiological patch clamp study to assess the potential effects of test compounds on human ether-a- go-go-related gene (hERG)-encoded channel tail current recorded from HEK293 cells stably transfected with hERG cDNA. Coverslips on which cells are seeded are mounted in a recording chamber and superfused with physiogical saline. Recordings of tail current are made in the voltage patch clamp mode. A reference substance e.g. E-4031 is used to confirm that the current observed can be inhibited by a known hERG channel blocker (Zhou, Z. et al., Biophys. J., 1998, 74, 230-241).

Compounds of the current invention typically show weak hERG channel affinities. Generally, the hERG channel affinity of compounds of formula (I) is greater than or equal to 1 μM.

### EXAMPLE 8

### Brain H₃ Receptors Occupancy

Material and Methods

Reagents

[³H]-N-α-methylhistamine (80-85 Ci/mmol) is purchased from Perkin Elmer (Belgium). Reagents and reference compounds used for binding assay on cerebral cortical tissues are of analytical grade and obtained from various commercial sources. Reference compounds are dissolved in 100% dimethylsulfoxide (DMSO) to give a 1 mM stock solution. Final DMSO concentration in the assay does not exceed 1%.

Animals and Treatments

Experimental procedures involving animals are conducted in compliance with the local ethics committee for animal experimentation according to Belgian law. Young male SPF Sprague-Dawley rats (OFA origin, supplied by IFFA CREDO, Belgium) weighting 200-300 g are used. Animals receive vehicle or the test compound by the i.p. route of administration. Compounds are all dissolved in a mixture of methyl cellulose (MC) 1% and DMSO 5%. A dose-volume of 5 ml/kg body weight is used. Control groups receive an equivalent dose-volume of MC 1% / DMSO 5%. Animals are killed one or three hours later. Terminal blood samples are collected and brains rapidly removed. Cerebral cortex are dissected on ice at 4° C.

Membrane Preparation

Cerebral cortex tissues are rapidly homogenized in 2.5 volumes of ice-cold buffer containing 50 mM Tris-HCl and 250 mM sucrose (pH 7.4). Homogenates are frozen in liquid nitrogen and stored at −80° C. until use.

³H]-N-α-methylhistamine Binding Assay

³H]-N-α-methylhistamine binding assay is carried out in 50 mM Tris-HCl buffer (pH 7.4) containing 2 mM MgCl₂. Briefly, homogenates are thawed and incubated for 15 minutes at room temperature before use. Homogenates (500 μg of proteins) are incubated at 25° C. during 5 minutes in 0.2 ml of buffer and 0.2 nM [³H]-N-α-methylhistamine. Non specific binding (NSB) is defined as the residual binding observed in the presence of 10 μM thioperamide. Membrane-bound and free radioligand are separated by rapid filtration through glass fiber filters (GF/C) (pre-soaked in 0.1% PEI). Samples and filters are rinsed by 8 ml of ice-cold 50 mM Tris-HCl buffer (pH 7.4). The entire filtration procedure does not exceed 10 seconds per sample. Radioactivity trapped onto the filters is counted by liquid scintillation in a R-counter. Protein concentrations are determined using the BCA Pierce method with bovine serum albumin as a standard.

Data Analysis

Percentage of receptor occupancy was defined as:

1-((B-NSB)/(B-NSB)_{(treated animals)}/(B-NSB)_{(control animals)}))·100

wherein B is the radioligand bound (dpm) and NSB is the non specific binding.

IC₅₀ values (dose required to produce a 50% reduction in ex vivo radioligand binding) are determined by plotting and analyzing the log₁₀ of the i.p. dose against % specific binding by non-linear regression using GraphPad Prism 4 software (GraphPad Inc., San Diego, USA) according to the following generic equation:

Y=MIN+(MAX−MIN)/(1+10^{(LogIC}50^{-X)*nH)})

wherein Y is the response, X is the logarithm of the concentration, MIN is the minimal binding observed (dpm), MAX is maximal binding observed (dpm) and nH is the Hill coefficient.

Preferred compounds of formula (I) according to the present invention typically show a percentage of receptor occupancy generally greater than or equal to 70% at a dose of 1 mg/kg ip.

1. A compound of formula (I), a geometrical isomer, an enantiomer, a diastereoisomer, a pharmaceutically acceptable salt or mixture thereof, 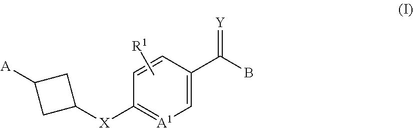
wherein
A is a substituted or unsubstituted amino group which is linked to the cyclobutyl group via an amino nitrogen;
A¹ is CH, C-halogen, C-alkoxy or N;
Y is O or S;
B is a substituted or unsubstituted amino group which is linked to carbonyl or thiocarbonyl group via an amino nitrogen;
X is O or S; and
R¹ is hydrogen or C₁₋₆ alkyl or halogen or C₁₋₆ alkoxy; and
with the proviso that compound of formula (I) is different from N-(2-oxoazepan-3-yl)-4-{[trans-3-(piperidin-1-yl)cyclobutyl]oxy}benzamide; and
with the proviso that B is an amino group different from —NH₂; and
with the proviso that when X ix O and Y is O, B is different from a group of formula (IX), 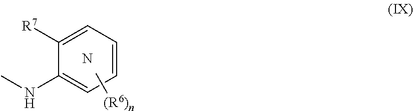
wherein R⁶ is selected from the group comprising or consisting of sulfonyl, amino, substituted or unsubstituted C₁₋₆ alkyl, substituted or unsubstituted C₂₋₆ alkenyl, substituted or unsubstituted C₂₋₆ alkynyl, substituted or unsubstituted aryl, substituted or unsubstituted heteroaryl, substituted or unsubstituted C₃₋₈ cycloalkyl, substituted or unsubstituted 3-8-membered heterocycloalkyl, acyl, substituted or unsubstituted C₁₋₆-alkyl aryl, substituted or unsubstituted C₁₋₆-alkyl heteroaryl, substituted or unsubstituted C₂₋₆-alkenyl aryl, substituted or unsubstituted C₂₋₆-alkenyl heteroaryl, substituted or unsubstituted C₂₋₆-alkynyl aryl, substituted or unsubstituted C₂₋₆-alkynyl heteroaryl, substituted or unsubstituted C₁₋₆-alkyl cycloalkyl, substituted or unsubstituted C₁₋₆-alkyl heterocycloalkyl, substituted or unsubstituted C₂₋₆-alkenyl cycloalkyl, substituted or unsubstituted C₂₋₆-alkenyl heterocycloalkyl, substituted or unsubstituted C₂₋₆-alkynyl cycloalkyl, substituted or unsubstituted C₂₋₆-alkynyl heterocycloalkyl, alkoxycarbonyl, aminocarbonyl, substituted or unsubstituted C₁₋₆-alkyl carboxy, substituted or unsubstituted C₁₋₆-alkyl acyl, substituted or unsubstituted aryl acyl, substituted or unsubstituted heteroaryl acyl, substituted or unsubstituted C₃₋₈-(hetero)cycloalkyl acyl, substituted or unsubstituted C₁₋₆-alkyl acyloxy, substituted or unsubstituted C₁₋₆-alkyl alkoxy, substituted or unsubstituted C₁₋₆-alkyl alkoxycarbonyl, substituted or unsubstituted C₁₋₆-alkyl aminocarbonyl, substituted or unsubstituted C₁₋₆-alkyl acylamino, acylamino, acylaminocarbonyl, ureido, substituted or unsubstituted C₁₋₆-alkyl ureido, substituted or unsubstituted C₁₋₆-alkyl carbamate, substituted or unsubstituted C₁₋₆-alkyl amino, substituted or unsubstituted C₁₋₆-alkyl sulfonyloxy, substituted or unsubstituted C₁₋₆-alkyl sulfonyl, substituted or unsubstituted C₁₋₆-alkyl sulfinyl, substituted or unsubstituted C₁₋₆-alkyl sulfanyl, substituted or unsubstituted C₁₋₆-alkyl sulfonylamino, aminosulfonyl, substituted or unsubstituted C₁₋₆-alkyl aminosulfonyl, hydroxy, substituted or unsubstituted C₁₋₆-alkyl hydroxy, phosphonate, substituted or unsubstituted C₁₋₆-alkyl phosphonate, halogen, cyano, carboxy, oxo and thioxo;
R⁷ is Cl or NH₂; and
n is equal to 0, 1, 2 or 3.

2. A compound of formula (I), a geometrical isomers, an enantiomer, a diastereoisomer, pharmaceutically acceptable salt or a possible mixture thereof, 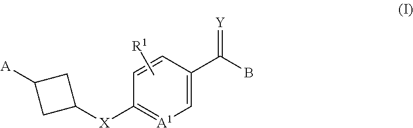
wherein
A is a substituted or unsubstituted amino group which is linked to the cyclobutyl group via an amino nitrogen;
A¹ is CH, C-halogen, C-alkoxy or N;
Y is O or S;
B is a substituted or unsubstituted cyclic amino group which is linked to carbonyl or thiocarbonyl group via an amino nitrogen;
X is O or S; and
R¹ is hydrogen or C₁₋₆ alkyl or halogen or C₁₋₆ alkoxy.

3. The compound of formula (Ia) according to claim 1, wherein the moieties A and X are in a trans configuration to each other, pharmaceutically acceptable salts and all possible mixtures thereof, 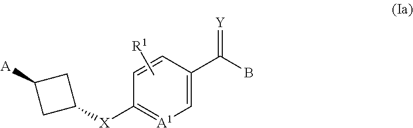
wherein A, A¹, B, X, Y and R¹ are as herein defined for compound of formula (I).

4. The compound according to claim 1 wherein A¹ is CH, C—F, C—Cl, C—O—CH₃ or N.

5. The compound according to claim 1 wherein A¹ is CH.

6. The compound according to claim 1 wherein X is O.

7. The compound according to claim 1 wherein Y is O.

8. The compound according to claim 1 wherein A is a 3 to 8 membered heterocycloalkyl linked to the cyclobutyl group via a nitrogen atom.

9. The compound according to claim 1 wherein A is a 3 to 8 membered heterocycloalkyl selected from the groups comprising or consisting of substituted or unsubstituted piperidin-1-yl, substituted or unsubstituted morpholin-4-yl, substituted or unsubstituted pyrrolidin-1-yl, substituted or unsubstituted piperazin-1-yl, substituted or unsubstituted azepan-1-yl or substituted or unsubstituted thiomorpholin-4-yl.

10. The compound according to claim 1 wherein A is a 3 to 8 membered heterocycloalkyl selected from the groups comprising or consisting of substituted or unsubstituted piperidin-1-yl or substituted or unsubstituted pyrridin-1-yl.

11. The compound according to claim 1 wherein B is a 3 to 8 membered heterocycloalkyl linked to the carbonyl or thiocarbonyl group via a nitrogen atom.

12. The compound according to claim 1 wherein B is a 3 to 8 membered heterocycloalkyl selected from the groups comprising or consisting of substituted or unsubstituted piperidin-1-yl, substituted or unsubstituted morpholin-4-yl, substituted or unsubstituted pyrrolidin-1-yl, substituted or unsubstituted piperazin-1-yl, substituted or unsubstituted thiomorpholin-4-yl, substituted or unsubstituted azetidin-1-yl, substituted or unsubstituted imidazolidin-1-yl, substituted or unsubstituted isoxazolidin-2-yl, substituted or unsubstituted 1,3-thiazolidin-3-yl, substituted or unsubstituted pyrazolidin-1-yl and substituted or unsubstituted 1,4-oxazepan-4-yl.

13. The compound according to claim 1 wherein B is a group of formula —NR⁴R⁵ wherein R⁴ and R⁵ are independently hydrogen, C₁₋₆ alkyl, aryl or heteroaryl, provided that at least one of R⁴ and R⁵ is different from hydrogen.

14. The compound of formula (Ib) according to claim 2, geometrical isomers, enantiomers, diastereoisomers, pharmaceutically acceptable salts and all possible mixtures thereof, 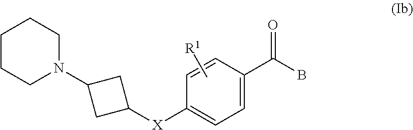
wherein X and R¹ are as herein defined for formula (I); and
B is a substituted or unsubstituted 3-8 membered heterocycloalkyl which is linked to the carbonyl via an amino nitrogen.

15. The compound of formula (Ic) according to claim 2, geometrical isomers, enantiomers, diastereoisomers, pharmaceutically acceptable salts and all possible mixtures thereof, 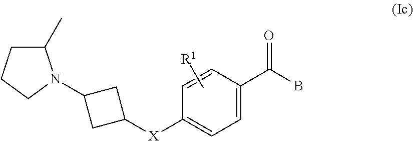
wherein X and R¹ are as herein defined for formula (I); and
B is a substituted or unsubstituted 3-8 membered heterocycloalkyl which is linked to the carbonyl via an amino nitrogen.

16. The compound according to claim 14 wherein B is a 3 to 8 membered heterocycloalkyl selected from substituted or unsubstituted piperidin-1-yl, substituted or unsubstituted morpholin-4-yl, substituted or unsubstituted pyrrolidin-1-yl or substituted or unsubstituted 1,4-oxazepan-4-yl.

17. The compound according to claim 14 wherein B is a 3 to 8 membered heterocycloalkyl selected from substituted or unsubstituted piperidin-1-yl or substituted or unsubstituted morpholin-4-yl.

18. The compound according to claim 1 wherein R¹ is hydrogen, methoxy, chlorine or fluorine.

19. A compound according to claim 1 wherein R¹ is hydrogen.

20. The compound according to claim 1 which is
4-{4-[(trans-3-piperidin-1-ylcyclobutyl)oxy]benzoyl}morpholine;
4,4-difluoro-1-{4-[(trans-3-piperidin-1-ylcyclobutyl)oxy]benzoyl}piperidine;
1-{trans-3-[4-(piperidin-1-ylcarbonyl)phenoxy]cyclobutyl}piperidine;
1-isopropyl-4-{4-[(trans-3-piperidin-1-ylcyclobutyl)oxy]benzoyl}piperazine;
1-{4-[(trans-3-piperidin-1-ylcyclobutyl)oxy]benzoyl}piperidin-4-ol;
4-{4-[(trans-3-piperidin-1-ylcyclobutyl)oxy]benzoyl}thiomorpholine;
1-{trans-3-[4-(pyrrolidin-1-ylcarbonyl)phenoxy]cyclobutyl}piperidine;
4-{4-[(trans-3-piperidin-1-ylcyclobutyl)oxy]benzoyl}thiomorpholine 1,1-dioxide;
1-(trans-3-{4-[(3,3-difluoropyrrolidin-1-yl)carbonyl]phenoxy}cyclobutyl)piperidine;
1-acetyl-4-{4-[(trans-3-piperidin-1-ylcyclobutyl)oxy]benzoyl}piperazine; and
1-{4-[(trans-3-piperidin-1-ylcyclobutyl)oxy]benzoyl}azetidin-3-ol;
4-[(trans-3-piperidin-1-ylcyclobutyl)oxy]-N-(2,2,2-trifluoroethyl)benzamide;
4-[4-({trans-3-[(2R)-2-methylpyrrolidin-1-yl]cyclobutyl}oxy)benzoyl]morpholine; 4-[4-({trans-3-[(2S)-2-methylpyrrolidin-1-yl]cyclobutyl}oxy)benzoyl]morpholine;
4-(4-{[trans-3-(2-methylpyrrolidin-1-yl)cyclobutyl]oxy}benzoyl)morpholine;
4-{[4-({trans-3-[(2S)-2-methylpyrrolidin-1-yl]cyclobutyl}oxy)phenyl]carbonothioyl}morpholine;
4-{3-chloro-4-[(trans-3-piperidin-1-ylcyclobutyl)oxy]benzoyl}morpholine;
1-{2-chloro-4-[(trans-3-piperidin-1-ylcyclobutyl)oxy]benzoyl}imidazolidin-4-one;
4-{2-fluoro-4-[(trans-3-piperidin-1-ylcyclobutyl)oxy]benzoyl}morpholine;
1-[trans-3-(3-chloro-4-{[2-(methoxymethyl)pyrrolidin-1-yl]carbonyl}phenoxy)cyclobutyl]piperidine;
2-{3-chloro-4-[(trans-3-piperidin-1-ylcyclobutyl)oxy]benzoyl}isoxazolidin-4-ol;
1-{trans-3-[3-chloro-4-(1,3-thiazolidin-3-ylcarbonyl)phenoxy]cyclobutyl}piperidine;
1-{4-[(trans-3-piperidin-1-ylcyclobutyl)oxy]benzoyl}pyrazolidin-3-one;
2-{4-[(trans-3-piperidin-1-ylcyclobutyl)oxy]benzoyl}isoxazolidin-4-ol;
4-{3-chloro-4-[(trans-3-piperidin-1-ylcyclobutyl)oxy]benzoyl}-1,4-oxazepane;
1-{4-[(trans-3-piperidin-1-ylcyclobutyl)oxy]benzoyl}piperidine-4-carboxamide;
1-{trans-3-[3-chloro-4-(pyrrolidin-1-ylcarbonyl)phenoxy]cyclobutyl}piperidine;
4-{2-fluoro-4-[(trans-3-piperidin-1-ylcyclobutyl)oxy]benzoyl}-1,4-oxazepane;
1-[trans-3-(4-{[2-(methoxymethyl)pyrrolidin-1-yl]carbonyl}phenoxy)cyclobutyl]piperidine;
4-{2-chloro-4-[(trans-3-piperidin-1-ylcyclobutyl)oxy]benzoyl}-1,4-oxazepane;
2-{2-chloro-4-[(trans-3-piperidin-1-ylcyclobutyl)oxy]benzoyl}isoxazolidin-4-ol;
1-{3-fluoro-4-[(trans-3-piperidin-1-ylcyclobutyl)oxy]benzoyl}piperidin-4-one;
1-{trans-3-[4-(1,3-thiazolidin-3-ylcarbonyl)phenoxy]cyclobutyl}piperidine;
1-{2-chloro-4-[(trans-3-piperidin-1-ylcyclobutyl)oxy]benzoyl}piperidine-4-carboxamide;
1-{4-[(trans-3-piperidin-1-ylcyclobutyl)oxy]benzoyl}imidazolidin-4-one;
1-[trans-3-(3-fluoro-4-{[2-(methoxymethyl)pyrrolidin-1-yl]carbonyl}phenoxy)cyclobutyl]piperidine;
1-{3-methoxy-4-[(trans-3-piperidin-1-ylcyclobutyl)oxy]benzoyl}imidazolidin-4-one;
4-{4-[(trans-3-piperidin-1-ylcyclobutyl)oxy]benzoyl}-1,4-oxazepane;
4-{2-chloro-4-[(trans-3-piperidin-1-ylcyclobutyl)oxy]benzoyl}morpholine;
1-{trans-3-[3-fluoro-4-(1,3-thiazolidin-3-ylcarbonyl)phenoxy]cyclobutyl}piperidine;
4-{4-[(trans-3-piperidin-1-ylcyclobutyl)thio]benzoyl}morpholine; or
1-{4-[(trans-3-piperidin-1-ylcyclobutyl)thio]benzoyl}piperidin-4-one.

21. A composition comprising a compound of formula (I), or a pharmaceutically acceptable salt thereof in combination with a pharmaceutically acceptable diluent or carrier.

22. (canceled)

23. A method of treating of mild-cognitive impairement, Alzheimer's disease, learning and memory disorders, attention-deficit hyperactivity disorder, Parkinson's disease, schizophrenia, dementia, depression, epilepsy, seizures, convulsions, sleep/wake disorders, cognitive dysfunctions, narcolepsy, hypersomnia, obesity, upper airway allergic disorders, Down's syndrome, anxiety, stress, cardiovascular disorders, inflammation, pain disorders, particularly neuropathic pain, or multiple sclerosis, comprising administering an effective amount of a compound according to claim 2 to a subject in need thereof.

24. A compound which is
1-[trans-3-(4-bromo-2-fluorophenoxy)cyclobutyl]piperidine;
1-[trans-3-(4-bromo-2-methoxyphenoxy)cyclobutyl]piperidine;
1-[trans-3-(4-bromo-2-chlorophenoxy)cyclobutyl]piperidine;
1-[trans-3-(4-bromo-3-fluorophenoxy)cyclobutyl]piperidine;
1-[trans-3-(4-bromo-3-chlorophenoxy)cyclobutyl]piperidine;
4-{[trans-3-(piperidin-1-yl)cyclobutyl]oxy}benzoic acid;
3-fluoro-4-{[trans-3-(piperidin-1-yl)cyclobutyl]oxy}benzoic acid;
3-methoxy-4-{[trans-3-(piperidin-1-yl)cyclobutyl]oxy}benzoic acid;
3-chloro-4-{[trans-3-(piperidin-1-yl)cyclobutyl]oxy}benzoic acid;
2-fluoro-4-{[trans-3-(piperidin-1-yl)cyclobutyl]oxy}benzoic acid; or
2-chloro-4-{[trans-3-(piperidin-1-yl)cyclobutyl]oxy}benzoic acid.

25. The compound of formula (Ia) according to claim 2, wherein the moieties A and X are in a trans configuration to each other, pharmaceutically acceptable salts and all possible mixtures thereof, 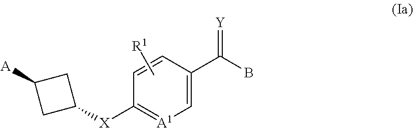
wherein A, A¹, B, X, Y and R¹ are as herein defined for compound of formula (I).

26. The compound according to claim 2 wherein A is a 3 to 8 membered heterocycloalkyl linked to the cyclobutyl group via a nitrogen atom.

27. The compound according to claim 2 wherein A is a 3 to 8 membered heterocycloalkyl selected from the groups comprising or consisting of substituted or unsubstituted piperidin-1-yl, substituted or unsubstituted morpholin-4-yl, substituted or unsubstituted pyrrolidin-1-yl, substituted or unsubstituted piperazin-1-yl, substituted or unsubstituted azepan- 1-yl or substituted or unsubstituted thiomorpholin-4-yl.

28. The compound according to claim 2 wherein A is a 3 to 8 membered heterocycloalkyl selected from the groups comprising or consisting of substituted or unsubstituted piperidin-1-yl or substituted or unsubstituted pyrridin-1-yl.

29. The compound according to claim 2 wherein B is a 3 to 8 membered heterocycloalkyl linked to the carbonyl or thiocarbonyl group via a nitrogen atom.

30. The compound according to claim 2 wherein B is a group of formula —NR⁴R⁵ wherein R⁴ and R⁵ are independently hydrogen, C₁₋₆ alkyl, aryl or heteroaryl, provided that at least one of R⁴ and R⁵ is different from hydrogen.

31. The compound according to any claim 2 wherein B is a 3 to 8 membered heterocycloalkyl selected from the groups comprising or consisting of substituted or unsubstituted piperidin-1-yl, substituted or unsubstituted morpholin-4-yl, substituted or unsubstituted pyrrolidin-1-yl, substituted or unsubstituted piperazin-1-yl, substituted or unsubstituted thiomorpholin-4-yl, substituted or unsubstituted azetidin-1-yl, substituted or unsubstituted imidazolidin-1-yl, substituted or unsubstituted isoxazolidin-2-yl, substituted or unsubstituted 1,3-thiazolidin-3-yl, substituted or unsubstituted pyrazolidin-1-yl and substituted or unsubstituted 1,4-oxazepan-4-yl.

32. The compound according to claim 2 wherein R¹ is hydrogen, methoxy, chlorine or fluorine.

33. A compound according to any preceding claim 2 wherein R¹ is hydrogen.

34. The compound according to claim 2 which is
4-{4-[(trans-3-piperidin-1-ylcyclobutyl)oxy]benzoyl}morpholine;
4,4-difluoro-1-{4-[(trans-3-piperidin-1-ylcyclobutyl)oxy]benzoyl}piperidine;
1-{trans-3-[4-(piperidin-1-ylcarbonyl)phenoxy]cyclobutyl}piperidine;
1-isopropyl-4-{4-[(trans-3-piperidin-1-ylcyclobutyl)oxy]benzoyl}piperazine;
1-{4-[(trans-3-piperidin-1-ylcyclobutyl)oxy]benzoyl}piperidin-4-ol;
4-{4-[(trans-3-piperidin-1-ylcyclobutyl)oxy]benzoyl}thiomorpholine;
1-{trans-3-[4-(pyrrolidin-1-ylcarbonyl)phenoxy]cyclobutyl}piperidine;
4-{4-[(trans-3-piperidin-1-ylcyclobutyl)oxy]benzoyl}thiomorpholine 1,1-dioxide;
1-(trans-3-{4-[3,3-difluoropyrrolidin-1-yl)carbonyl]phenoxy}cyclobutyl)piperidine;
1-acetyl-4-{4-[(trans-3-piperidin-1-ylcyclobutyl)oxy]benzoyl}piperazine; and
1-{4-[(trans-3-piperidin-1-ylcyclobutyl)oxy]benzoyl}azetidin-3-ol;
4-[(trans-3-piperidin-1-ylcyclobutyl)oxy]-N-(2,2,2-trifluoroethyl)benzamide;
4-[4-({trans-3-[(2R)-2-methylpyrrolidin-1-yl]cyclobutyl}oxy)benzoyl]morpholine; 4-[4-({trans-3-[(2S)-2-methylpyrrolidin-1-yl]cyclobutyl}oxy)benzoyl]morpholine;
4-(4-{[trans-3-(2-methylpyrrolidin-1-yl)cyclobutyl]oxy}benzoyl)morpholine;
4-{[4-({trans-3-[(2S)-2-methylpyrrolidin-1-yl]cyclobutyl}oxy)phenyl]carbonothioyl}morpholine;
4-{3-chloro-4-[(trans-3-piperidin-1-ylcyclobutyl)oxy]benzoyl}morpholine;
1-{2-chloro-4-[(trans-3-piperidin-1-ylcyclobutyl)oxy]benzoyl}imidazolidin-4-one;
4-{2-fluoro-4-[(trans-3-piperidin-1-ylcyclobutyl)oxy]benzoyl}morpholine;
1-[trans-3-(3-chloro-4-{[2-(methoxymethyl)pyrrolidin-1-yl]carbonyl}phenoxy)cyclobutyl]piperidine;
2-{3-chloro-4-[(trans-3-piperidin-1-ylcyclobutyl)oxy]benzoyl}isoxazolidin-4-ol;
1-{trans-3-[3-chloro-4-(1,3-thiazolidin-3-ylcarbonyl)phenoxy]cyclobutyl}piperidine;
1-{4-[(trans-3-piperidin-1-ylcyclobutyl)oxy]benzoyl}pyrazolidin-3-one;
2-{4-[(trans-3-piperidin-1-ylcyclobutyl)oxy]benzoyl}isoxazolidin-4-ol;
4-{3-chloro-4-[(trans-3-piperidin-1-ylcyclobutyl)oxy]benzoyl}-1,4-oxazepane;
1-{4-[(trans-3-piperidin-1-ylcyclobutyl)oxy]benzoyl}piperidine-4-carboxamide;
1-{trans-3-[3-chloro-4-(pyrrolidin-1-ylcarbonyl)phenoxy]cyclobutyl}piperidine;
4-{2-fluoro-4-[(trans-3-piperidin-1-ylcyclobutyl)oxy]benzoyl}-1,4-oxazepane;
1-[trans-3-(4-{[2-(methoxymethyl)pyrrolidin-1-yl]carbonyl}phenoxy)cyclobutyl]piperidine;
4-{2-chloro-4-[(trans-3-piperidin-1-ylcyclobutyl)oxy]benzoyl}-1,4-oxazepane;
2-{2-chloro-4-[(trans-3-piperidin-1-ylcyclobutyl)oxy]benzoyl}isoxazolidin-4-ol;
1-{3-fluoro-4-[(trans-3-piperidin-1-ylcyclobutyl)oxy]benzoyl}piperidin-4-one;
1-{trans-3-[4-(1,3-thiazolidin-3-ylcarbonyl)phenoxy]cyclobutyl}piperidine;
1-{2-chloro-4-[(trans-3-piperidin-1-ylcyclobutyl)oxy]benzoyl}piperidine-4-carboxamide;
1-{4-[(trans-3-piperidin-1-ylcyclobutyl)oxy]benzoyl}imidazolidin-4-one;
1-[trans-3-(3-fluoro-4-{[2-(methoxymethyl)pyrrolidin-1-yl]carbonyl}phenoxy)cyclobutyl]piperidine;
1-{3-methoxy-4-[(trans-3-piperidin-1-ylcyclobutyl)oxy]benzoyl}imidazolidin-4-one;
4-{4-[(trans-3-piperidin-1-ylcyclobutyl)oxy]benzoyl}-1,4-oxazepane;
4-{2-chloro-4-[(trans-3-piperidin-1-ylcyclobutyl)oxy]benzoyl}morpholine;
1-{trans-3-[3-fluoro-4-(1,3-thiazolidin-3-ylcarbonyl)phenoxy]cyclobutyl}piperidine;
4-{4-[(trans-3-piperidin-1-ylcyclobutyl)thio]benzoyl}morpholine; and
1-{4-[(trans-3-piperidin-1-ylcyclobutyl)thio]benzoyl}piperidin-4-one.
